(19) United States
(12) Patent Application Publication
(10) Pub. No.: US 2024/0410802 A1
(43) Pub. Date: Dec. 12, 2024

(54) ANALYSIS METHOD FOR MINIMUM CROSS-SECTION CENTER STRESS AND STRAIN OF TENSILE SPECIMEN WITH NECKING DEFORMATION

(71) Applicant: Luoyang Ship Material Research Institute, Luoyang (CN)

(72) Inventors: Gang XUE, Luoyang (CN); Wei TANG, Luoyang (CN); Zhenpeng GAO, Luoyang (CN); Xinyao ZHANG, Luoyang (CN)

(21) Appl. No.: 18/737,613

(22) Filed: Jun. 7, 2024

(30) Foreign Application Priority Data
Jun. 9, 2023 (CN) 202310683056.0

(51) Int. Cl. G01N 3/08; G16C 60/00

(52) U.S. Cl. CPC G01N 3/08; G16C 60/00; G01N 2203/0003; G01N 2203/0017; G01N 2203/0075; G01N 2203/0252; G01N 2203/0266; G01N 2203/0676; G01N 2203/0682

(57) ABSTRACT

Analysis method for minimum necking deformation cross-section center stress and strain comprising: recording values for axial acting force, minimum cross-section radius, maximum limit of cross-section radius, inflection point position tangent slope of a rotational generatrix of a contour, radius of cross-section perpendicular to central axis at the inflection point position, and distance between the cross-section perpendicular to the central axis at the inflection point position and minimum cross-section at a necking bottom; and establishing a rectangular coordinate system by taking a center position of the minimum cross-section at the necking bottom as an origin, and substituting the recorded values into mathematical models of a first, second, and third principal stress at a center position of the minimum cross-section to calculate and obtain three principal stress values at the center position of the minimum cross-section, e.g., to calculate stress components, equivalent stresses (Mises), stress invariants, and equivalent plastic strain.

### CROSS-REFERENCE TO RELATED APPLICATIONS

This application claims priority to Chinese Application No. 202310683056.0 filed on Jun. 9, 2023, the contents of which are incorporated fully herein by reference.

### TECHNICAL FIELD

The present invention relates to the technical field of material tests, and in particular, relates to an analysis method for minimum cross-section center stress and strain of a tensile specimen with necking deformation.

### BACKGROUND

The uniaxial tensile test of round bar specimens is one of the most fundamental methods for testing the mechanical properties of materials, and the test can determine parameters such as the yield strength, tensile strength, reduction of area, and elongation after fracture of metal materials. Necking deformation is a common phenomenon in the uniaxial tensile test of round bar specimens for metal materials like low-alloy steel, and this phenomenon is characterized by the specimen transitioning into a state of plastic deformation concentrated in part of the region after undergoing a certain degree of uniform plastic deformation. As the necking deformation destroys the mechanical state of the previous uniaxial stress of the specimen, the stress-strain field within the necking deformation region cannot be determined by adopting a calculation method under the uniaxial stress state. The center of the minimum cross-section at the necking bottom during the necking deformation stage is a maximum value region for the equivalent plastic strain and is also the position where the fracture is generated firstly, so an analysis method for stress and strain at a center position of a minimum cross-section of a necking bottom during necking deformation stage is established, which is of great significance to measure the stress-strain constitutive relation and the fracture strength of metal materials under the condition of large plastic strain by adopting a uniaxial tensile test of round bar specimens.

Bridgeman establishes calculation methods for internal stress and strain of a cross-section at a necking bottom on the premise that the contour of the necking bottom is circular and the rheological stresses σ₀ within the minimum cross-section at the necking bottom are equal; namely, the calculation equation of the equivalent strain ε̅ is

$$\overline{ε} = 2 ln \frac{{r}_{0}}{r} ,$$

and the calculation equation of the rheological stress σ₀ is

$${σ}_{0} = \frac{F}{π \cdot {r}^{2} \cdot ( 1 + \frac{2 R}{r} ) \cdot ln ( 1 + \frac{r}{2 R} )} ,$$

wherein in the equations, r is the radius of the minimum cross-section at the necking bottom, r₀ is the initial value of the radius of the minimum cross-section at the necking bottom, R is the arc radius of the specimen contour at the minimum cross-section position of the necking bottom, and F is the acting force. Patents CN108982222A and CN108982223A propose a method for constructing an interpolation curve expression of a necking contour by adopting a necking step model respectively for a metal plate-shaped specimen and a metal round bar specimen to determine the arc radius R and the minimum cross-section radius r of the specimen contour at the minimum cross-section position of the necking bottom, and further determining the stress and strain during the necking deformation stage by adopting a Bridgeman calculation method. Patent CN109883824A proposes a method for inversely calculating the arc radius R and the minimum cross-section radius r of the specimen contour at the minimum cross-section position of the necking bottom at each moment by measuring the gauge length elongation and the coordinate information of the outer contour curve of the broken specimen during the tensile process of a round bar specimen, and further determining the stress and strain during the necking deformation stage by adopting a Bridgeman calculation method. Patent CN113281171A proposes a method for measuring the arc radius R and the minimum cross-section radius r of the specimen contour at the minimum cross-section position of the necking bottom by means of an optical microscope by collecting the tensile load and contour images of the necking region in real time during the test process, and further determining the stress and strain during the necking deformation stage by adopting a Bridgeman calculation method. Chen Chi et al. established a calculation method for internal stress and strain of the minimum cross-section at the necking bottom that is similar to the Bridgeman calculation method on the premise that the contour of the necking bottom meets the distribution of a hyperbolic function and the rheological stresses σ₀ within the minimum cross-section at the necking bottom are equal; namely, the calculation equation of the equivalent strain ε̅ is

$$\overline{ε} = 2 ln \frac{{r}_{0}}{r} ,$$

and the calculation equation of the rheological stress σ₀ is

$${σ}_{0} = \frac{F}{π \cdot {r}^{2} \cdot ( 1 + \frac{2 R}{r} ) \cdot ln ( 1 + \frac{r}{2 R} )} ,$$

wherein in the equations, r is the radius of the minimum cross-section at the necking bottom, r₀ is the initial value of the radius of the minimum cross-section at the necking bottom, R is the curvature radius of the specimen contour at the minimum cross-section position of the necking bottom, and F is the acting force. Patent CN109883823A proposes a method for inversely calculating the arc radius R and the minimum cross-section radius r of the specimen contour at the minimum cross-section position of the necking bottom at each moment by measuring the gauge length elongation and the coordinate information of the outer contour curve of the broken specimen during the tensile process of a round bar specimen, and further determining the stress and strain during the necking deformation stage by adopting the Chen Chi's calculation method. However, the prerequisite of the Bridgeman calculation method is that the contour of the necking bottom is circular, and the rheological stresses σ₀ within the minimum cross-section at the necking bottom are equal; the prerequisite of the Chen Chi's calculation method is that the contour of the necking bottom meets the distribution of a hyperbolic function, and the rheological stresses σ₀ within the minimum cross-section at the necking bottom are equal. In fact, according to the research in “Correlation Analysis of Deformation Characteristics and Stress-Strain Constitutive Relation of Isotropic Homogeneous Elastoplastic Material Round Bar Specimens in Uniaxial Tensile Test”, the contour of the necking bottom is S-shaped, and the rheological stresses σ₀ at different positions on the minimum cross-section at the necking bottom are not equal, so the prerequisites of the Bridgeman calculation method and the Chen Chi's calculation method are not the same as the actual situation of the test, and thus the stress and strain at the center of the minimum cross-section during the necking stage cannot be accurately analyzed.

Patent CN101975693A proposes a method for determining the true stress and true strain of materials by measuring and calculating engineering stress and engineering strain of a round bar specimen under different reference gauge lengths during the uniaxial tensile test process of the specimen and calculating by adopting a conversion equation of uniaxial stress uniform deformation, but this method ignores that as the necking region is in a triaxial stress state, the stress-strain calculation method for a uniaxial stress uniform deformation state is not suitable for stress-strain calculation in the triaxial stress state. Patent CN103175735A proposes a method for determining a material stress-strain constitutive relation curve with a finite element simulation result identical to a test measurement result by measuring the contour curve and the stress-strain field data of a round bar specimen during the necking deformation stage in the uniaxial tensile test, comparing the finite element simulation result with the test measurement result, and using an iterative solution method, where the curve is a true stress-strain constitutive relation curve of the material, and the method has high accuracy but relatively low efficiency due to a large amount of finite element simulation work needed in one test.

### SUMMARY

In view of this, the present invention aims to provide an analysis method for minimum cross-section center stress and strain of a round bar specimen during a necking deformation stage in a uniaxial tensile test, so as to achieve the objective of measuring the minimum cross-section center stress and strain of the metal material round bar specimen during the necking deformation stage in the uniaxial tensile test by constructing corresponding mathematical models.

Disclosed in the present invention is an analysis method for minimum cross-section center stress and strain of a tensile specimen with necking deformation, which is used for detecting and analyzing a round bar specimen and includes the following steps:
S1: performing a uniaxial tensile test, recording a tensile axial acting force F_{z} in a test process in real time, recording a change situation of a diameter of the specimen, and acquiring at least a radius r_{c} of a minimum cross-section at a necking bottom perpendicular to a central axis on the specimen, a maximum limit value rₙ of a radius of a cross-section perpendicular to the central axis on the specimen, a tangent slope kₜ^{ip} at an inflection point position of a rotational generatrix of a necking deformation contour, a radius rᵢₚ of a cross-section perpendicular to the central axis at the inflection point position of the rotational generatrix of the necking deformation contour, and a distance zᵢₚ between the cross-section perpendicular to the central axis at the inflection point position of the rotational generatrix of the necking deformation contour and the minimum cross-section at the necking bottom;
S2: setting a hypothetical condition according to characteristics of a contour line of necking deformation, establishing a rectangular coordinate system by taking a center position of the minimum cross-section at the necking bottom perpendicular to the central axis as an origin, taking the central axis as a coordinate z-axis, and taking any two radius lines perpendicular to each other and intersecting at the circle center within the minimum cross-section at the necking bottom as an x-axis and a y-axis of the coordinate system; and
S3: according to F_{z}, r_{c}, rₙ, kₜ^{ip}, rᵢₚ, and zᵢₚ, acquired in S1, performing a calculation on a first principal stress σ₁^{c}, a second principal stress σ₂^{c}, and a third principal stress σ₃^{c} at a center position of the minimum cross-section of the necking bottom based on equations (1) and (2),

$$\begin{matrix}{σ}_{1}^{c} = [ \begin{matrix}{k}_{0}^{z} + {k}_{11}^{z} \cdot {k}_{t}^{ip} + {k}_{21}^{z} \cdot \frac{{r}_{c}}{{z}_{ip}} + {k}_{31}^{z} \cdot \frac{{r}_{ip}}{{z}_{ip}} + {k}_{41}^{z} \cdot \frac{{r}_{n}}{{z}_{ip}} + \\ {k}_{12}^{z} \cdot {( {k}_{t}^{ip} )}^{2} + {k}_{22}^{z} \cdot {( \frac{{r}_{c}}{{z}_{ip}} )}^{2} + {k}_{32}^{z} \cdot {( \frac{{r}_{ip}}{{z}_{ip}} )}^{2} + {k}_{42}^{z} \cdot {( \frac{{r}_{n}}{{z}_{ip}} )}^{2} + \\ {k}_{13}^{z} \cdot {( {k}_{t}^{ip} )}^{3} + {k}_{23}^{z} \cdot {( \frac{{r}_{c}}{{z}_{ip}} )}^{3} + {k}_{33}^{z} \cdot {( \frac{{r}_{ip}}{{z}_{ip}} )}^{3} + {k}_{43}^{z} \cdot {( \frac{{r}_{n}}{{z}_{ip}} )}^{3}\end{matrix} ] \cdot \frac{{F}_{z}}{π \cdot {r}_{c}^{2}} & ( 1 ) \\ {σ}_{2}^{c} = {σ}_{3}^{c} = [ \begin{matrix}{k}_{0}^{x} + {k}_{11}^{x} \cdot {k}_{t}^{ip} + {k}_{21}^{x} \cdot \frac{{r}_{c}}{{z}_{ip}} + {k}_{31}^{x} \cdot \frac{{r}_{ip}}{{z}_{ip}} + {k}_{41}^{x} \cdot \frac{{r}_{n}}{{z}_{ip}} + \\ {k}_{12}^{x} \cdot {( {k}_{t}^{ip} )}^{2} + {k}_{22}^{x} \cdot {( \frac{{r}_{c}}{{z}_{ip}} )}^{2} + {k}_{32}^{x} \cdot {( \frac{{r}_{ip}}{{z}_{ip}} )}^{2} + {k}_{42}^{x} \cdot {( \frac{{r}_{n}}{{z}_{ip}} )}^{2} + \\ {k}_{13}^{x} \cdot {( {k}_{t}^{ip} )}^{3} + {k}_{23}^{x} \cdot {( \frac{{r}_{c}}{{z}_{ip}} )}^{3} + {k}_{33}^{x} \cdot {( \frac{{r}_{ip}}{{z}_{ip}} )}^{3} + {k}_{43}^{x} \cdot {( \frac{{r}_{n}}{{z}_{ip}} )}^{3}\end{matrix} ] \cdot \frac{{F}_{z}}{π \cdot {r}_{c}^{2}} & ( 2 )\end{matrix}$$

wherein σ₁^{c} is a positive stress component along the z-axis, σ₂^{c} is a positive stress component along the x-axis, σ₃^{c} is a positive stress component along the y-axis, and k₀^{z}, k₁₁^{z}, k₂₁^{z}, k₃₁^{z}, k₄₁^{z}, k₁₂^{z}, k₂₂^{z}, k₃₂^{z}, k₄₂^{z}, k₁₃^{z}, k₂₃^{z}, k₃₃^{z}, k₄₃^{z}, k₀^{x}, k₁₁^{x}, k₂₁^{x}, k₃₁^{x}, k₄₁^{x}, k₁₂^{x}, k₂₂^{x}, k₃₂^{x}, k₄₂^{x}, k₁₃^{x}, k₂₃^{x}, k₃₃^{x}, k₄₃^{x} are stress regression coefficients.

Further, the hypothetical condition in S2 is that: during a necking stage of the uniaxial tensile test of the round bar specimen, a shape of the specimen is a rotational body formed by rotating the rotational generatrix of the contour around the central axis; the specimen is symmetrical along a central axis direction with respect to the minimum cross-section at the necking bottom.

Further, k₀^{z}=1.087, k₁₁^{z}=−2.216, k₂₁^{z}=−5.935, k₃₁^{z}=5.144, k₄₁^{z}=0.432, k₁₂^{z}=0.761, k₂₁^{z}=1.828, k₃₂^{z}=−1.142, k₄₂^{z}=−0.233, k₁₃^{z}=−0.288, k₂₃^{z}=−0.483, k₃₃^{z}=0.336, k₄₃^{z}=0.026, k₀^{x}=0.059, k₁₁^{x}=−5.039, k₂₁^{x}=−11.289, k₃₁^{x}=10.229, k₄₁^{x}=0.791, k₁₂^{x}=2.439, k₂₂^{x}=2.634, k₃₂^{x}=−1.943, k₄₂^{x}=−0.397, k₁₃^{x}=−1.781, k₂₃^{x}=−0.624, k₃₃^{x}=0.565, k₄₃^{x}=0.043, and mathematical models of the first principal stress σ₁^{c}, the second principal stress σ₂^{c}, and the third principal stress σ₃^{c} obtained therefrom are shown in equations (3) and (4),

$${σ}_{1}^{c} = [ \begin{matrix}1.087 - 2.216 \cdot {k}_{t}^{ip} - 5.935 \cdot \frac{{r}_{c}}{{z}_{ip}} + 5.144 \cdot \frac{{r}_{ip}}{{z}_{ip}} + 0.432 \cdot \frac{{r}_{n}}{{z}_{ip}} + \\ 0.761 \cdot {( {k}_{t}^{ip} )}^{2} + 1.828 \cdot {( \frac{{r}_{c}}{{z}_{ip}} )}^{2} - 1.142 \cdot {( \frac{{r}_{ip}}{{z}_{ip}} )}^{2} - 0.233 \cdot {( \frac{{r}_{n}}{{z}_{ip}} )}^{2} - \\ 0.288 \cdot {( {k}_{t}^{ip} )}^{3} - 0.483 \cdot {( \frac{{r}_{c}}{{z}_{ip}} )}^{3} + 0.336 \cdot {( \frac{{r}_{ip}}{{z}_{ip}} )}^{3} + 0.026 \cdot {( \frac{{r}_{n}}{{z}_{ip}} )}^{3}\end{matrix} ] \cdot \frac{{F}_{z}}{π \cdot {r}_{c}^{2}} \qquad ( 3 )$$

$${σ}_{2}^{c} = {σ}_{3}^{c} = [ \begin{matrix}0.059 - 5.039 \cdot {k}_{t}^{ip} - 11.289 \cdot \frac{{r}_{c}}{{z}_{ip}} + 10.229 \cdot \frac{{r}_{ip}}{{z}_{ip}} + 0.791 \cdot \frac{{r}_{n}}{{z}_{ip}} + \\ 2.439 \cdot {( {k}_{t}^{ip} )}^{2} + 2.634 \cdot {( \frac{{r}_{c}}{{z}_{ip}} )}^{2} - 1.943 \cdot {( \frac{{r}_{ip}}{{z}_{ip}} )}^{2} - 0.397 \cdot {( \frac{{r}_{n}}{{z}_{ip}} )}^{2} - \\ 1.781 \cdot {( {k}_{t}^{ip} )}^{3} - 0.624 \cdot {( \frac{{r}_{c}}{{z}_{ip}} )}^{3} + 0.565 \cdot {( \frac{{r}_{ip}}{{z}_{ip}} )}^{3} + 0.043 \cdot {( \frac{{r}_{n}}{{z}_{ip}} )}^{3}\end{matrix} ] \cdot \frac{{F}_{z}}{π \cdot {r}_{c}^{2}} . \qquad ( 4 )$$

Further, the analysis method further includes:
calculating a stress first invariant I₁^{c} at the center position of the minimum cross-section of the necking bottom based on equations (1) and (2) in S3 as shown in equation (5)

$${I}_{1}^{c} = {σ}_{1}^{c} + {σ}_{2}^{c} + {σ}_{3}^{c} \qquad ( 5 )$$

and/or,
calculating a Mises equivalent stress σ̅_̅{̅c̅}̅ at the center position of the minimum cross-section of the necking bottom based on equations (1) and (2) in S3 as shown in equation (6)

$$\overline{{σ}_{c}} = \sqrt{\frac{{( {σ}_{1}^{c} - {σ}_{2}^{c} )}^{2} + {( {σ}_{1}^{c} - {σ}_{3}^{c} )}^{2} + {( {σ}_{2}^{c} - {σ}_{3}^{c} )}^{2}}{2}} . \qquad ( 6 )$$

Further, k₀^{z}=1.087, k₁₁^{z}=−2.216, k₂₁^{z}=−5.935, k₃₁^{z}=5.144, k₄₁^{z}=0.432, k₁₂^{z}=0.761, k₂₂^{z}=1.828, k₃₂^{z}=−1.142, k₄₂^{z}=−0.233, k₁₃^{z}=−0.288, k₂₃^{z}=−0.483, k₃₃^{z}=0.336, k₄₃^{z}=0.026, k₀^{x}=0.059, k₁₁^{x}=−5.039, k₂₁^{x}=−11.289, k₃₁^{x}=10.229, k₄₁^{x}=0.791, k₁₂^{x}=2.439, k₂₂^{x}=2.634, k₃₂^{x}=−1.943, k₄₂^{x}=−0.397, k₁₃^{x}=−1.781, k₂₃^{x}=−0.624, k₃₃^{x}=0.565, k₄₃^{x}=0.043, a mathematical model of the stress first invariant I₁^{c} at the center position of the minimum cross-section of the necking bottom obtained therefrom is shown in equation (7), and/or, a mathematical model of the Mises equivalent stress σ̅_̅{̅C̅}̅ at the center position of the minimum cross-section of the necking bottom obtained is shown in equation (8),

$${I}_{1}^{c} = [ \begin{matrix}1.206 - 12.294 \cdot {k}_{t}^{ip} - 28.513 \cdot \frac{{r}_{c}}{{z}_{ip}} + 25.602 \cdot \frac{{r}_{ip}}{{z}_{ip}} + 2.013 \cdot \frac{{r}_{n}}{{z}_{ip}} + \\ 5.638 \cdot {( {k}_{t}^{ip} )}^{2} + 7.095 \cdot {( \frac{{r}_{c}}{{z}_{ip}} )}^{2} - 5.027 \cdot {( \frac{{r}_{ip}}{{z}_{ip}} )}^{2} - 1.026 \cdot {( \frac{{r}_{n}}{{z}_{ip}} )}^{2} - \\ 3.85 \cdot {( {k}_{t}^{ip} )}^{3} - 1.731 \cdot {( \frac{{r}_{c}}{{z}_{ip}} )}^{3} + 1.466 \cdot {( \frac{{r}_{ip}}{{z}_{ip}} )}^{3} + 0.113 \cdot {( \frac{{r}_{n}}{{z}_{ip}} )}^{3}\end{matrix} ] \cdot \frac{{F}_{z}}{π \cdot {r}_{c}^{2}} \qquad ( 7 )$$

$$\overline{{σ}_{c}} = [ \begin{matrix}1.028 + 2.823 \cdot {k}_{t}^{ip} + 5.354 \cdot \frac{{r}_{c}}{{z}_{ip}} - 5.085 \cdot \frac{{r}_{ip}}{{z}_{ip}} - 0.359 \cdot \frac{{r}_{n}}{{z}_{ip}} - \\ 1.678 \cdot {( {k}_{t}^{ip} )}^{2} - 0.806 \cdot {( \frac{{r}_{c}}{{z}_{ip}} )}^{2} + 0.801 \cdot {( \frac{{r}_{ip}}{{z}_{ip}} )}^{2} + 0.164 \cdot {( \frac{{r}_{n}}{{z}_{ip}} )}^{2} + \\ 1.493 \cdot {( {k}_{t}^{ip} )}^{3} + 0.141 \cdot {( \frac{{r}_{c}}{{z}_{ip}} )}^{3} - 0.229 \cdot {( \frac{{r}_{ip}}{{z}_{ip}} )}^{3} - 0.018 \cdot {( \frac{{r}_{n}}{{z}_{ip}} )}^{3}\end{matrix} ] \cdot \frac{{F}_{z}}{π \cdot {r}_{c}^{2}} . \qquad ( 8 )$$

Further, the following step is performed before S1:
S0: measuring an initial cross-section radius R_{c} of the specimen before the test; on the basis of S0, S1 further includes:
S11: acquiring a maximum value F_{z}^{max} of an acting force in a central axis direction and a minimum cross-section radius r_{c}⁰ of the necking bottom at the moment; on the basis of S11, the analysis method further includes:
S4: according to F_{z}, r_{c}, rₙ, kₜ^{ip}, rᵢₚ, zᵢₚ, F_{z}^{max}, and r_{c}⁰ obtained in S1, performing a calculation on equivalent plastic strain at the center of the minimum cross-section of necking based on equation (9),

$$\overline{{ε}_{p}^{c}} = 2 ln \frac{{R}_{c}}{{r}_{c}^{0}} - \frac{{F}_{z}^{max}}{π \cdot {( {r}_{c}^{0} )}^{2} \cdot E} + {k}_{0}^{\overline{ε}} + {k}_{11}^{\overline{ε}} \cdot {k}_{t}^{ip} + {k}_{21}^{\overline{ε}} \cdot \frac{{r}_{c}}{{z}_{ip}} + {k}_{31}^{\overline{ε}} \cdot \frac{{r}_{ip}}{{z}_{ip}} + {k}_{41}^{\overline{ε}} \cdot \frac{{r}_{n}}{{z}_{ip}} + {k}_{12}^{\overline{ε}} \cdot {( {k}_{t}^{ip} )}^{2} + {k}_{22}^{\overline{ε}} \cdot {( \frac{{r}_{c}}{{z}_{ip}} )}^{2} + {k}_{32}^{\overline{ε}} \cdot {( \frac{{r}_{ip}}{{z}_{ip}} )}^{2} + {k}_{42}^{\overline{ε}} \cdot {( \frac{{r}_{n}}{{z}_{ip}} )}^{2} + {k}_{13}^{\overline{ε}} \cdot {( {k}_{t}^{ip} )}^{3} + {k}_{23}^{\overline{ε}} \cdot {( \frac{{r}_{c}}{{z}_{ip}} )}^{3} + {k}_{33}^{\overline{ε}} \cdot {( \frac{{r}_{ip}}{{z}_{ip}} )}^{3} + {k}_{43}^{\overline{ε}} \cdot {( \frac{{r}_{n}}{{z}_{ip}} )}^{3} \qquad ( 9 )$$

wherein ε̅ₚ̅^̅{̅c̅}̅ is the equivalent plastic strain at the center of the minimum cross-section of necking, E is an elastic modulus of a tensile specimen material, and k₀^{ε̅}, k₁₁^{ε̅}, k₂₁^{ε̅}, k₃₁^{ε̅}, k₄₁^{ε̅}, k₁₂^{ε̅}, k₂₂^{ε̅}, k₃₂^{ε̅}, k₄₂^{ε̅}, k₁₃^{ε̅}, k₂₃^{ε̅}, k₃₃^{ε̅}, and k₄₃^{ε̅} are equivalent strain regression coefficients.

Further, k₀^{ε̅}=0.108, k₁₁^{ε̅}=−0.396, k₂₁^{ε̅}=−9.768, k₃₁^{ε̅}=6.479, k₄₁^{ε̅}=2.978, k₁₂^{ε̅}=−1.193, k₂₂^{ε̅}=4.659, k₃₂^{ε̅}=−3.650, k₄₂^{ε̅}=−0.732, k₁₃^{ε̅}=1.699, k₂₃^{ε̅}=−1.076, k₃₃^{ε̅}=0.926, k₄₃^{ε̅}=0.068, and a mathematical model of the equivalent plastic strain ε̅ₚ̅^̅{̅c̅}̅ at the center of the minimum cross-section of necking obtained therefrom is shown in equation (10),

$$\overline{{ε}_{p}^{c}} = 2 ln \frac{{R}_{c}}{{r}_{c}^{0}} - \frac{{F}_{z}^{max}}{π \cdot {( {r}_{c}^{0} )}^{2} \cdot E} + 0.108 - 0.396 \cdot {k}_{t}^{ip} - 9.768 \cdot \frac{{r}_{c}}{{z}_{ip}} + 6.479 \cdot \frac{{r}_{ip}}{{z}_{ip}} + 2.978 \cdot \frac{{r}_{n}}{{z}_{ip}} - 1.193 \cdot {( {k}_{t}^{ip} )}^{2} + 4.659 \cdot {( \frac{{r}_{c}}{{z}_{ip}} )}^{2} - 3.65 \cdot {( \frac{{r}_{ip}}{{z}_{ip}} )}^{2} - 0.732 \cdot {( \frac{{r}_{n}}{{z}_{ip}} )}^{2} + 1.699 \cdot {( {k}_{t}^{ip} )}^{3} - 1.076 \cdot {( \frac{{r}_{c}}{{z}_{ip}} )}^{3} + 0.926 \cdot {( \frac{{r}_{ip}}{{z}_{ip}} )}^{3} + 0.068 \cdot {( \frac{{r}_{n}}{{z}_{ip}} )}^{3} . \qquad ( 10 )$$

Further, the following step is performed before S1:
S0′: measuring an initial cross-section radius R_{c} of the specimen before the test;
on the basis of S0′, S1 further includes:

S11′: acquiring a maximum value F_{z}^{max} of an acting force in a central axis direction and a minimum cross-section radius r_{c}⁰ of the necking bottom at the moment;
on the basis of S11′, the analysis method further includes:
S4: according to F_{z}, r_{c}, rₙ, kₜ^{ip}, rᵢₚ, zᵢₚ, and r_{c}⁰ obtained in S1, performing a calculation on equivalent plastic strain at the center of the minimum cross-section of necking based on equation (11),

$$\overline{{ε}_{p}^{c}} = 2 ln \frac{{R}_{c}}{{r}_{c}^{0}} + {k}_{0}^{\overline{ε}} + {k}_{11}^{\overline{ε}} \cdot {k}_{t}^{ip} + {k}_{21}^{\overline{ε}} \cdot \frac{{r}_{c}}{{z}_{ip}} + {k}_{31}^{\overline{ε}} \cdot \frac{{r}_{ip}}{{z}_{ip}} + {k}_{41}^{\overline{ε}} \cdot \frac{{r}_{n}}{{z}_{ip}} + {k}_{12}^{\overline{ε}} \cdot {( {k}_{t}^{ip} )}^{2} + {k}_{22}^{\overline{ε}} \cdot {( \frac{{r}_{c}}{{z}_{ip}} )}^{2} + {k}_{32}^{\overline{ε}} \cdot {( \frac{{r}_{ip}}{{z}_{ip}} )}^{2} + {k}_{42}^{\overline{ε}} \cdot {( \frac{{r}_{n}}{{z}_{ip}} )}^{2} + {k}_{13}^{\overline{ε}} \cdot {( {k}_{t}^{ip} )}^{3} + {k}_{23}^{\overline{ε}} \cdot {( \frac{{r}_{c}}{{z}_{ip}} )}^{3} + {k}_{33}^{\overline{ε}} \cdot {( \frac{{r}_{ip}}{{z}_{ip}} )}^{3} + {k}_{43}^{\overline{ε}} \cdot {( \frac{{r}_{n}}{{z}_{ip}} )}^{3} \qquad ( 11 )$$

wherein ε̅ₚ̅^̅{̅c̅}̅ is the equivalent plastic strain at the center of the minimum cross-section of necking, and k₀^{ε̅}, k₁₁^{ε̅}, k₂₁^{ε̅}, k₃₁^{ε̅}, k₄₁^{ε̅}, k₁₂^{ε̅}, k₂₂^{ε̅}, k₃₂^{ε̅}, k₄₂^{ε̅}, k₁₃^{ε̅}, k₂₃^{ε̅}, k₃₃^{ε̅}, and k₄₃^{ε̅} are equivalent strain regression coefficients.

Further, k₀^{ε̅}=0.108, k₁₁^{ε̅}=−0.396, k₂₁^{ε̅}=−9.768, k₃₁^{ε̅}=6.479, k₄₁^{ε̅}=2.978, k₁₂^{ε̅}=−1.193, k₂₂^{ε̅}=4.659, k₃₂^{ε̅}=−3.650, k₄₂^{ε̅}=−0.732, k₁₃^{ε̅}=1.699, k₂₃^{ε̅}=−1.076, k₃₃^{ε̅}=0.926, k₄₃^{ε̅}=0.068, and a mathematical model of the equivalent plastic strain ε̅ₚ̅^̅{̅c̅}̅ at the center of the minimum cross-section of necking obtained therefrom is shown in equation (12),

$$\overline{{ε}_{p}^{c}} = 2 ln \frac{{R}_{c}}{{r}_{c}^{0}} + 0.108 - 0.396 \cdot {k}_{t}^{ip} - 9.768 \cdot \frac{{r}_{c}}{{z}_{ip}} + 6.479 \cdot \frac{{r}_{ip}}{{z}_{ip}} + 2.978 \cdot \frac{{r}_{n}}{{z}_{ip}} - 1.193 \cdot {( {k}_{t}^{ip} )}^{2} + 4.659 \cdot {( \frac{{r}_{c}}{{z}_{ip}} )}^{2} - 3.65 \cdot {( \frac{{r}_{ip}}{{z}_{ip}} )}^{2} - 0.732 \cdot {( \frac{{r}_{n}}{{z}_{ip}} )}^{2} + 1.699 \cdot {( {k}_{t}^{ip} )}^{3} - 1.076 \cdot {( \frac{{r}_{c}}{{z}_{ip}} )}^{3} + 0.926 \cdot {( \frac{{r}_{ip}}{{z}_{ip}} )}^{3} + 0.068 \cdot {( \frac{{r}_{n}}{{z}_{ip}} )}^{3} . \qquad ( 12 )$$

Further, in S1, a specimen shape image is acquired during the necking deformation stage and dimension measurement and calculation are performed to obtain shape characteristic parameters, and the shape characteristic parameters obtained by measurement and calculation at least include the radius r_{c} of the minimum cross-section at the necking bottom perpendicular to the central axis on the specimen, the maximum limit value rₙ of the radius of the cross-section perpendicular to the central axis on the specimen, the tangent slope kₜ^{ip} at the inflection point position of the rotational generatrix of the necking deformation contour, the radius rᵢₚ of the cross-section perpendicular to the central axis at the inflection point position of the rotational generatrix of the necking deformation contour, and the distance zᵢₚ between the cross-section perpendicular to the central axis at the inflection point position of the rotational generatrix of the necking deformation contour and the minimum cross-section at the necking bottom.

Compared with the prior art, the analysis method for minimum cross-section center stress and strain of a tensile specimen with necking deformation of the present invention has the following advantages:

The analysis method provided in the present invention can effectively calculate and determine each principal stress component, a Mises equivalent stress, a stress first invariant, and equivalent plastic strain of the minimum cross-section center at the necking bottom during the necking deformation stage in the uniaxial tensile test of the round bar specimen. The analysis method provided in the present invention has the advantages of a clear physical mechanism, a concise mathematical model, and high analytical precision.

### BRIEF DESCRIPTION OF THE DRAWINGS

In order to more clearly illustrate the technical solutions in the embodiments of the present invention or in the prior art, the drawings required to be used in the description of the embodiments or the prior art are briefly introduced below. It is obvious that the drawings in the description below are merely some embodiments of the present invention, and those of ordinary skills in the art can obtain other drawings according to these drawings without creative efforts.

FIG. 1 is a schematic diagram of the specimen shape of a round bar specimen during the necking deformation stage in a uniaxial tensile test according to an embodiment of the present invention;

FIG. 2 is a schematic diagram illustrating the construction of the specimen shape and the rectangular coordinate system of a round bar specimen during the uniaxial tensile necking deformation stage according to an embodiment of the present invention;

FIG. 3 is a schematic diagram of the shape characteristic parameters of the specimen during the necking deformation stage according to an embodiment of the present invention;

FIG. 4 is a schematic diagram illustrating the comparison between fitted values of σ_{z}^{c}/σ_{z̅}ⁿ and finite element analysis values of σ_{z}^{c}/σ_{z̅}ⁿ for accuracy according to an embodiment of the present invention;

FIG. 5 is a schematic diagram illustrating the comparison between fitted values of σₓ^{c}/σ_{z̅}ⁿ and finite element analysis values of σₓ^{c}/σ_{z̅}ⁿ for accuracy according to an embodiment of the present invention;

FIG. 6 is a schematic diagram illustrating the comparison between derived values of I₁^{c} and finite element analysis values of I₁^{c} for accuracy according to an embodiment of the present invention;

FIG. 7 is a schematic diagram illustrating the comparison between derived values of σ̅_̅{̅c̅}̅ and finite element analysis values of σ̅_̅{̅c̅}̅ for accuracy according to an embodiment of the present invention;

FIG. 8 is a schematic diagram illustrating the comparison between fitted values of ε̅ₚ̅^̅{̅c̅}̅ and finite element analysis values of ε̅ₚ̅^̅{̅c̅}̅ for accuracy according to an embodiment of the present invention;

FIG. 9 is a schematic diagram illustrating the comparison between approximately calculated values of ε̅ₚ̅^̅{̅c̅}̅ and finite element analysis values of ε̅ₚ̅^̅{̅c̅}̅ for accuracy according to an embodiment of the present invention; and

FIG. 10 is a schematic diagram illustrating the comparison between approximately calculated values of ε̅ₚ̅^̅{̅c̅}̅ and finite element analysis values of ε̅ₚ̅^̅{̅c̅}̅ for accuracy in the prior art.

### DETAILED DESCRIPTION

To make the technical means of the present invention and its objectives and effects easy to understand, the following detailed description of the embodiments of the present invention is provided in conjunction with specific illustrations.

It should be noted that all terms indicating direction and position in the present invention, such as “up”, “down”, “left”, “right”, “front”, “back”, “vertical”, “horizontal”, “inner”, “outer”, “top”, “bottom”, “transverse”, “longitudinal”, and “center”, are used solely to explain the relative positional relationships and connection situations between the components in a specific state (as shown in the drawings). These terms are intended merely for the convenience of describing the present invention and do not necessitate that the present invention be constructed or operated in a specific orientation. Therefore, these terms should not be construed as limiting the present invention. In addition, in the present invention, descriptions involving “first”, “second”, etc., are used for the description purpose only and should not be understood as indicating or implying relative importance or implicitly specifying the quantity of the indicated technical features.

In the description of the present invention, unless otherwise explicitly specified and defined, the terms “install”, “connect”, and “link” should be understood in a broad sense. For example, they may refer to fixed connections, detachable connections, or integral connections; they may be mechanical connections; they may be directly connected or indirectly connected through an intermediary medium, and they may refer to the internal communication between two elements. For those of ordinary skill in the art, the specific meanings of the aforementioned terms in the present invention can be understood according to specific conditions.

In the specification, the reference term “an embodiment”, “some embodiments”, “illustrative embodiments”, “an example”, “a specific example”, or “some examples” means that a specific feature, structure, material, or characteristic described in connection with the embodiment or example is included in at least one embodiment or example of the present invention. In the specification, the schematic description of the aforementioned terms does not necessarily refer to the same embodiment or example. Moreover, the specific feature, structure, material, or characteristic described may be combined in a suitable manner in any one or more embodiments or examples.

Disclosed in the present invention is an analysis method for minimum cross-section center stress and strain of a tensile specimen with necking deformation, which is used for detecting and analyzing a round bar specimen and includes the following steps:
S1: performing a uniaxial tensile test, recording a tensile axial acting force F_{z} in a test process in real time, recording a change situation of a diameter of the specimen, and acquiring at least a radius r_{c} of a minimum cross-section at a necking bottom perpendicular to a central axis on the specimen, a maximum limit value rₙ of a radius of a cross-section perpendicular to the central axis on the specimen, a tangent slope kₜ^{ip} at an inflection point position of a rotational generatrix of a necking deformation contour, a radius rᵢₚ of a cross-section perpendicular to the central axis at the inflection point position of the rotational generatrix of the necking deformation contour, and a distance zᵢₚ between the cross-section perpendicular to the central axis at the inflection point position of the rotational generatrix of the necking deformation contour and the minimum cross-section at the necking bottom;
S2: setting a hypothetical condition according to characteristics of a contour line of necking deformation, establishing a rectangular coordinate system by taking a center position of the minimum cross-section at the necking bottom perpendicular to the central axis as an origin, taking the central axis as a coordinate z-axis, and taking any two radius lines perpendicular to each other and intersecting at the circle center within the minimum cross-section at the necking bottom as an x-axis and a y-axis of the coordinate system; and
S3: according to F_{z}, r_{c}, rₙ, kₜ^{ip}, rᵢₚ, and zᵢₚ acquired in S1, performing a calculation on a first principal stress σ₁^{c}, a second principal stress σ₂^{c}, and a third principal stress σ₃^{c} at a center position of the minimum cross-section of the necking bottom based on equations (1) and (2),

$$\begin{matrix}{σ}_{1}^{c} = [ \begin{matrix}{k}_{0}^{z} + {k}_{11}^{z} \cdot {k}_{t}^{ip} + {k}_{21}^{z} \cdot \frac{{r}_{c}}{{z}_{ip}} + {k}_{31}^{z} \cdot \frac{{r}_{ip}}{{z}_{ip}} + {k}_{41}^{z} \cdot \frac{{r}_{n}}{{z}_{ip}} + \\ {k}_{12}^{z} \cdot {( {k}_{t}^{ip} )}^{2} + {k}_{22}^{z} \cdot {( \frac{{r}_{c}}{{z}_{ip}} )}^{2} + {k}_{32}^{z} \cdot {( \frac{{r}_{ip}}{{z}_{ip}} )}^{2} + {k}_{42}^{z} \cdot {( \frac{{r}_{n}}{{z}_{ip}} )}^{2} + \\ {k}_{13}^{z} \cdot {( {k}_{t}^{ip} )}^{3} + {k}_{23}^{z} \cdot {( \frac{{r}_{c}}{{z}_{ip}} )}^{3} + {k}_{33}^{z} \cdot {( \frac{{r}_{ip}}{{z}_{ip}} )}^{3} + {k}_{43}^{z} \cdot {( \frac{{r}_{n}}{{z}_{ip}} )}^{3}\end{matrix} ] \cdot \frac{{F}_{z}}{π \cdot {r}_{c}^{2}} & ( 1 ) \\ {σ}_{2}^{c} = {σ}_{3}^{c} = [ \begin{matrix}{k}_{0}^{x} + {k}_{11}^{x} \cdot {k}_{t}^{ip} + {k}_{21}^{x} \cdot \frac{{r}_{c}}{{z}_{ip}} + {k}_{31}^{x} \cdot \frac{{r}_{ip}}{{z}_{ip}} + {k}_{41}^{x} \cdot \frac{{r}_{n}}{{z}_{ip}} + \\ {k}_{12}^{x} \cdot {( {k}_{t}^{ip} )}^{2} + {k}_{22}^{x} \cdot {( \frac{{r}_{c}}{{z}_{ip}} )}^{2} + {k}_{32}^{x} \cdot {( \frac{{r}_{ip}}{{z}_{ip}} )}^{2} + {k}_{42}^{x} \cdot {( \frac{{r}_{n}}{{z}_{ip}} )}^{2} + \\ {k}_{13}^{x} \cdot {( {k}_{t}^{ip} )}^{3} + {k}_{23}^{x} \cdot {( \frac{{r}_{c}}{{z}_{ip}} )}^{3} + {k}_{33}^{x} \cdot {( \frac{{r}_{ip}}{{z}_{ip}} )}^{3} + {k}_{43}^{x} \cdot {( \frac{{r}_{n}}{{z}_{ip}} )}^{3}\end{matrix} ] \cdot \frac{{F}_{z}}{π \cdot {r}_{c}^{2}} & ( 2 )\end{matrix}$$

wherein σ₁^{c} is a positive stress component along the z-axis, σ₂^{c} is a positive stress component along the x-axis, σ₃^{c} is a positive stress component along the y-axis, and k₀^{z}, k₁₁^{z}, k₂₁^{z}, k₃₁^{z}, k₄₁^{z}, k₁₂^{z}, k₂₂^{z}, k₃₂^{z}, k₄₂^{z}, k₁₃^{z}, k₂₃^{z}, k₃₃^{z}, k₄₃^{z}, k₀^{x}, k₁₁^{x}, k₂₁^{x}, k₃₁^{x}, k₄₁^{x}, k₁₂^{x}, k₂₂^{x}, k₃₂^{x}, k₄₂^{x}, k₁₃^{x}, k₂₃^{x}, k₃₃^{x}, k₄₃^{x} are stress regression coefficients.

By means of the establishment of the above mathematical models, corresponding parameters can be detected during the specimen tension process, then the stress distribution at the center position of the minimum cross-section of the necking portion can be accurately analyzed according to the parameters, and the stress first invariant and the Mises equivalent stress at the center of the minimum cross-section of the necking portion can be determined according to the stress distribution in the subsequent research process. In the prior art, there is no precedent that F_{z}, r_{c}, rₙ, kₜ^{ip}, rᵢₚ, and zᵢₚ, are used as independent variables to analyze the minimum cross-section center stress and strain of necking deformation, so the technical solutions in the present application propose the above-mentioned more accurate mathematical models on the basis of accurately defining the contour shape of the specimen during necking deformation, which is of great significance to measure the stress-strain constitutive relation and the fracture strength of metal materials under the condition of large plastic strain by adopting a uniaxial tensile test of round bar specimens.

In this example, by means of a lot of research, the present inventors analyzed the correlation between the shape characteristic parameters r_{c}, rₙ, kₜ^{ip}, rᵢₚ, zᵢₚ, and the ratios σ_{z}^{c}/σ_{z̅}ⁿ and σ_{z}^{c}/σ_{z̅}ⁿ of the central stress component of the minimum cross-section of necking deformation to the axial average stress during the necking stage of the tensile specimen, and constructed regression equations by taking kₜ^{ip}, r_{c}/zᵢₚ, rᵢₚ/zᵢₚ, rₙ/zᵢₚ, and respective power functions as independent variables to obtain regression equations (13) and (14),

$$\begin{matrix}\frac{{σ}_{z}^{c}}{{σ}_{\overline{z}}^{n}} = {k}_{0}^{z} + {k}_{11}^{z} \cdot {k}_{t}^{ip} + {k}_{21}^{z} \cdot \frac{{r}_{c}}{{z}_{ip}} + {k}_{31}^{z} \cdot \frac{{r}_{ip}}{{z}_{ip}} + {k}_{41}^{z} \cdot \frac{{r}_{n}}{{z}_{ip}} + {k}_{12}^{z} \cdot {( {k}_{t}^{ip} )}^{2} + {k}_{22}^{z} \cdot {( \frac{{r}_{c}}{{z}_{ip}} )}^{2} + {k}_{32}^{z} \cdot {( \frac{{r}_{ip}}{{z}_{ip}} )}^{2} + {k}_{42}^{z} \cdot {( \frac{{r}_{n}}{{z}_{ip}} )}^{2} + {k}_{13}^{z} \cdot {( {k}_{t}^{ip} )}^{3} + {k}_{23}^{z} \cdot {( \frac{{r}_{c}}{{z}_{ip}} )}^{3} + {k}_{33}^{z} \cdot {( \frac{{r}_{ip}}{{z}_{ip}} )}^{3} + {k}_{43}^{z} \cdot {( \frac{{r}_{n}}{{z}_{ip}} )}^{3} & ( 13 ) \\ \frac{{σ}_{x}^{c}}{{σ}_{\overline{z}}^{n}} = {k}_{0}^{x} + {k}_{11}^{x} \cdot {k}_{t}^{ip} + {k}_{21}^{x} \cdot \frac{{r}_{c}}{{z}_{ip}} + {k}_{31}^{x} \cdot \frac{{r}_{ip}}{{z}_{ip}} + {k}_{41}^{x} \cdot \frac{{r}_{n}}{{z}_{ip}} + {k}_{12}^{x} \cdot {( {k}_{t}^{ip} )}^{2} + {k}_{22}^{x} \cdot {( \frac{{r}_{c}}{{z}_{ip}} )}^{2} + {k}_{32}^{x} \cdot {( \frac{{r}_{ip}}{{z}_{ip}} )}^{2} + {k}_{42}^{x} \cdot {( \frac{{r}_{n}}{{z}_{ip}} )}^{2} + {k}_{13}^{x} \cdot {( {k}_{t}^{ip} )}^{3} + {k}_{23}^{x} \cdot {( \frac{{r}_{c}}{{z}_{ip}} )}^{3} + {k}_{33}^{x} \cdot {( \frac{{r}_{ip}}{{z}_{ip}} )}^{3} + {k}_{43}^{x} \cdot {( \frac{{r}_{n}}{{z}_{ip}} )}^{3} & ( 14 )\end{matrix}$$

wherein σ_{z}^{c}=σ₁^{c}, σₓ^{c}=σ₂^{c}=σ₃^{c}, σ_{z̅}ⁿ is the axial average stress, and σ_{z̅}ⁿ is calculated based on equation (15),

$${σ}_{\overline{z}}^{n} = \frac{{F}_{z}}{π \cdot {r}_{c}^{2}} \qquad ( 15 )$$

Equations (1) and (2) can be obtained from equations (13), (14) and (15). By means of the establishment of the above mathematical models, the stress state of the necking deformation portion can be analyzed and obtained by detecting the shape characteristic parameters and the loading load during the tensile test stage, which has a good guiding significance for measuring the stress-strain constitutive relation and the fracture strength of metal materials.

The hypothetical condition in S2 is that: during a necking stage of the uniaxial tensile test of the round bar specimen, a shape of the specimen is a rotational body formed by rotating the rotational generatrix of the contour around the central axis; the specimen is symmetrical along a central axis direction with respect to the minimum cross-section at the necking bottom.

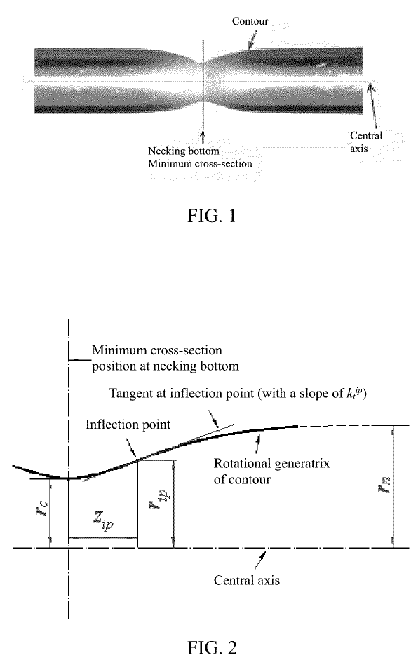

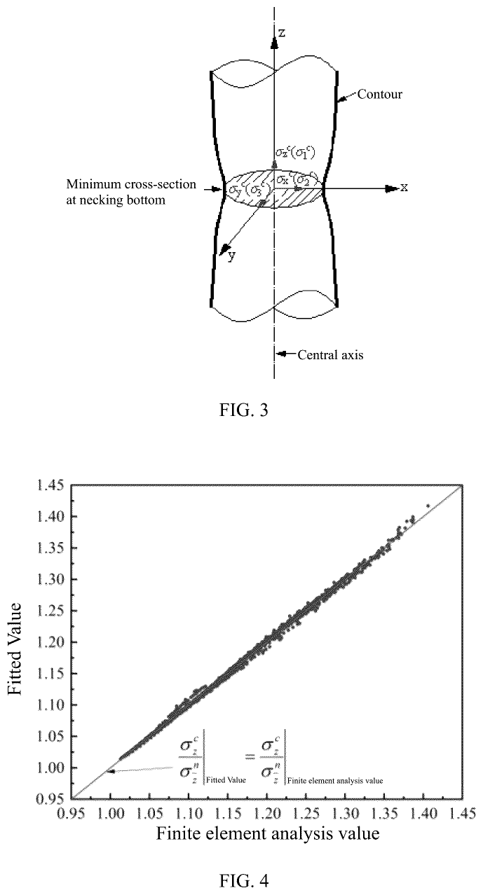

As shown in FIG. 1, during the necking stage in the uniaxial tensile test of the round bar specimen, the shape of the specimen is approximately a rotational body formed by rotating the rotational generatrix of the contour around the central axis. The rotational generatrix is a contour line shown in FIG. 1, and the specimen is symmetrical along the central axis direction with respect to the minimum cross-section at the necking bottom. The coordinate system established in step S2 is shown in FIG. 2, the contour line on one side of the cross-section is in an “S” shape, and the tangent of the contour line at the minimum cross-section position is parallel to the rotational axis.

As an example of the present invention, k₀^{z}=1.087, k₁₁^{z}=−2.216, k₂₁^{z}=−5.935, k₃₁^{z}=5.144, k₄₁^{z}=0.432, k₁₂^{z}=0.761, k₂₂^{z}=1.828, k₃₂^{z}=−1.142, k₄₂^{z}=−0.233, k₁₃^{z}=−0.288, k₂₃^{z}=−0.483, k₃₃^{z}=0.336, k₄₃^{z}=0.026, k₀^{x}=0.059, k₁₁^{x}=−5.039, k₂₁^{x}=−11.289, k₃₁^{x}=10.229, k₄₁^{x}=0.791, k₁₂^{x}=2.439, k₂₂^{x}=2.634, k₃₂^{x}=−1.943, k₄₂^{x}=−0.397, k₁₃^{x}=−1.781, k₂₃^{x}=−0.624, k₃₃^{x}=0.565, k₄₃^{x}=0.043, and mathematical models of the first principal stress σ₁^{c}, the second principal stress σ₂^{c}, and the third principal stress σ₃^{c} obtained therefrom are shown in equations (3) and (4),

$${σ}_{1}^{c} = [ \begin{matrix}1.087 - 2.216 \cdot {k}_{t}^{ip} - 5.935 \cdot \frac{{r}_{c}}{{z}_{ip}} + 5.144 \cdot \frac{{r}_{ip}}{{z}_{ip}} + 0.432 \cdot \frac{{r}_{n}}{{z}_{ip}} + \\ 0.761 \cdot {( {k}_{t}^{ip} )}^{2} + 1.828 \cdot {( \frac{{r}_{c}}{{z}_{ip}} )}^{2} - 1.142 \cdot {( \frac{{r}_{ip}}{{z}_{ip}} )}^{2} - 0.233 \cdot {( \frac{{r}_{n}}{{z}_{ip}} )}^{2} - \\ 0.288 \cdot {( {k}_{t}^{ip} )}^{3} - 0.483 \cdot {( \frac{{r}_{c}}{{z}_{ip}} )}^{3} + 0.336 \cdot {( \frac{{r}_{ip}}{{z}_{ip}} )}^{3} + 0.026 \cdot {( \frac{{r}_{n}}{{z}_{ip}} )}^{3}\end{matrix} ] \cdot \frac{{F}_{z}}{π \cdot {r}_{c}^{2}} \qquad ( 3 )$$

$${σ}_{2}^{c} = {σ}_{3}^{c} = [ \begin{matrix}0.059 - 5.036 \cdot {k}_{t}^{ip} - 11.289 \cdot \frac{{r}_{c}}{{z}_{ip}} + 10.229 \cdot \frac{{r}_{ip}}{{z}_{ip}} + 0.791 \cdot \frac{{r}_{n}}{{z}_{ip}} + \\ 2.439 \cdot {( {k}_{t}^{ip} )}^{2} + 2.634 \cdot {( \frac{{r}_{c}}{{z}_{ip}} )}^{2} - 1.943 \cdot {( \frac{{r}_{ip}}{{z}_{ip}} )}^{2} - 0.397 \cdot {( \frac{{r}_{n}}{{z}_{ip}} )}^{2} - \\ 1.781 \cdot {( {k}_{t}^{ip} )}^{3} - 0.624 \cdot {( \frac{{r}_{c}}{{z}_{ip}} )}^{3} + 0.565 \cdot {( \frac{{r}_{ip}}{{z}_{ip}} )}^{3} + 0.043 \cdot {( \frac{{r}_{n}}{{z}_{ip}} )}^{3}\end{matrix} ] \cdot \frac{{F}_{z}}{π \cdot {r}_{c}^{2}} . \qquad ( 4 )$$

The calculation equations of σ_{z}^{c}/σ_{z̅}ⁿ and σₓ^{c}/σ_{z̅}ⁿ corresponding to equations (3) and (4) are equation (16) and equation (17),

$$\begin{matrix}\frac{{σ}_{z}^{c}}{{σ}_{\overline{z}}^{n}} = 1.087 - 2.216 \cdot {k}_{t}^{ip} - 5.935 \cdot \frac{{r}_{c}}{{z}_{ip}} + 5.144 \cdot \frac{{r}_{ip}}{{z}_{ip}} + 0.432 \cdot \frac{{r}_{n}}{{z}_{ip}} + 0.761 \cdot {( {k}_{t}^{ip} )}^{2} + 1.828 \cdot {( \frac{{r}_{c}}{{z}_{ip}} )}^{2} - 1.142 \cdot {( \frac{{r}_{ip}}{{z}_{ip}} )}^{2} - 0.233 \cdot {( \frac{{r}_{n}}{{z}_{ip}} )}^{2} - 0.288 \cdot {( {h}_{t}^{ip} )}^{3} - 0.483 \cdot {( \frac{{r}_{c}}{{z}_{ip}} )}^{3} + 0.336 \cdot {( \frac{{r}_{ip}}{{z}_{ip}} )}^{3} + 0.026 \cdot {( \frac{{r}_{n}}{{z}_{ip}} )}^{3} & ( 16 ) \\ \frac{{σ}_{x}^{c}}{{σ}_{\overline{z}}^{n}} = 0.059 - 5.039 \cdot {k}_{t}^{ip} - 11.289 \cdot \frac{{r}_{c}}{{z}_{ip}} + 10.229 \cdot \frac{{r}_{ip}}{{z}_{ip}} + 0.791 \cdot \frac{{r}_{n}}{{z}_{ip}} + 2.439 \cdot {( {k}_{t}^{ip} )}^{2} + 2.634 \cdot {( \frac{{r}_{c}}{{z}_{ip}} )}^{2} - 1.943 \cdot {( \frac{{r}_{ip}}{{z}_{ip}} )}^{2} - 0.397 \cdot {( \frac{{r}_{n}}{{z}_{ip}} )}^{2} - 1.781 \cdot {( {h}_{t}^{ip} )}^{3} - 0.64 \cdot {( \frac{{r}_{c}}{{z}_{ip}} )}^{3} + 0.565 \cdot {( \frac{{r}_{ip}}{{z}_{ip}} )}^{3} + 0.043 \cdot {( \frac{{r}_{n}}{{z}_{ip}} )}^{3} . & ( 17 )\end{matrix}$$

Equations (13) and (14) are fitted by adopting finite element simulation data, wherein the coefficient of determination R² after fitting adjustment of equations (13) and (14) are 0.99809 and 0.99796, respectively, and the overall significance can both pass the F test. FIG. 4 shows the comparison between a fitted value of σ_{z}^{c}/σ_{z̅}ⁿ obtained from equation (16) and a finite element analysis value of σ_{z}^{c}/σ_{z̅}ⁿ.

It can be seen that all data points are distributed centrally around the straight line

$$\frac{{σ}_{z}^{c}}{{σ}_{\overline{z}}^{n}} \vert$$

### _{Fitted Value}

$$= \frac{{σ}_{z}^{c}}{{σ}_{\overline{z}}^{n}} \vert$$

_{Finite element analysis value}. FIG. 5 shows the comparison between a fitted value of σₓ^{c}/σ_{z̅}ⁿ obtained from equation (17) and a finite element analysis value of σₓ^{c}/σ_{z̅}ⁿ. It can be seen that all data points are distributed centrally around the straight line

$$\frac{{σ}_{x}^{c}}{{σ}_{\overline{z}}^{n}} \vert$$

### _{Fitted Value}

$$= \frac{{σ}_{x}^{c}}{{σ}_{\overline{z}}^{n}} \vert$$

_{Finite element analysis value}, which means that equation (16) and equation (17) have good fitting accuracy, and the stress component at the center of the minimum cross-section of necking deformation can be accurately calculated based on equation (3) and equation (4) obtained therefrom.

As an example of the present invention, the analysis method further includes:
calculating a stress first invariant I₁^{c} at the center position of the minimum cross-section of the necking bottom based on equations (1) and (2) in S3 as shown in equation (5)

$${I}_{1}^{c} = {σ}_{1}^{c} + {σ}_{2}^{c} + {σ}_{3}^{c} \qquad ( 5 )$$

and/or,
calculating a Mises equivalent stress σ̅_̅{̅c̅}̅ at the center position of the minimum cross-section of the necking bottom based on equations (1) and (2) in S3 as shown in equation (6)

$$\overline{{σ}_{c}} = \sqrt{\frac{{( {σ}_{1}^{c} - {σ}_{2}^{c} )}^{2} + {( {σ}_{1}^{c} - {σ}_{3}^{c} )}^{2} + {( {σ}_{2}^{c} - {σ}_{3}^{c} )}^{2}}{2}} . \qquad ( 6 )$$

In this example, k₀^{z}=1.087, k₁₁^{z}=−2.216, k₂₁^{z}=−5.935, k₃₁^{z}=5.144, k₄₁^{z}=0.432, k₁₂^{z}=0.761, k₂₂^{z}=1.828, k₃₂^{z}=−1.142, k₄₂^{z}=−0.233, k₁₃^{z}=−0.288, k₂₃^{z}=−0.483, k₃₃^{z}=0.336, k₄₃^{z}=0.026, k₀^{x}=0.059, k₁₁^{x}=−5.039, k₂₁^{x}=−11.289, k₃₁^{x}=10.229, k₄₁^{x}=0.791, k₁₂^{x}=2.439, k₂₂^{x}=2.634, k₃₂^{x}=−1.943, k₄₂^{x}=−0.397, k₁₃^{x}=−1.781, k₂₃^{x}=−0.624, k₃₃^{x}=0.565, k₄₃^{x}=0.043, a mathematical model of the stress first invariant I₁^{c} at the center position of the minimum cross-section of the necking bottom obtained therefrom is shown in equation (7), and/or, a mathematical model of the Mises equivalent stress σ̅_̅{̅c̅}̅ at the center position of the minimum cross-section of the necking bottom obtained is shown in equation (8),

$${I}_{1}^{c} = [ \begin{matrix}1.206 - 12.294 \cdot {k}_{t}^{ip} - 28.513 \cdot \frac{{r}_{c}}{{z}_{ip}} + 25.602 \cdot \frac{{r}_{ip}}{{z}_{ip}} + 2.013 \cdot \frac{{r}_{n}}{{z}_{ip}} + \\ 5.638 \cdot {( {k}_{t}^{ip} )}^{2} + 7.095 \cdot {( \frac{{r}_{c}}{{z}_{ip}} )}^{2} - 5.027 \cdot {( \frac{{r}_{ip}}{{z}_{ip}} )}^{2} - 1.206 \cdot {( \frac{{r}_{n}}{{z}_{ip}} )}^{2} - \\ 3.85 \cdot {( {k}_{t}^{ip} )}^{3} - 1.731 \cdot {( \frac{{r}_{c}}{{z}_{ip}} )}^{3} + 1.466 \cdot {( \frac{{r}_{ip}}{{z}_{ip}} )}^{3} + 0.113 \cdot {( \frac{{r}_{n}}{{z}_{ip}} )}^{3}\end{matrix} ] \cdot \frac{{F}_{z}}{π \cdot {r}_{c}^{2}} \qquad ( 7 )$$

$$\overline{{σ}_{c}} = [ \begin{matrix}1.028 + 2.823 \cdot {k}_{t}^{ip} + 5.354 \cdot \frac{{r}_{c}}{{z}_{ip}} - 5.805 \cdot \frac{{r}_{ip}}{{z}_{ip}} - 0.359 \cdot \frac{{r}_{n}}{{z}_{ip}} - \\ 1.678 \cdot {( {k}_{t}^{ip} )}^{2} - 0.806 \cdot {( \frac{{r}_{c}}{{z}_{ip}} )}^{2} + 0.801 \cdot {( \frac{{r}_{ip}}{{z}_{ip}} )}^{2} + 0.164 \cdot {( \frac{{r}_{n}}{{z}_{ip}} )}^{2} + \\ 1.493 \cdot {( {k}_{t}^{ip} )}^{3} + 0.141 \cdot {( \frac{{r}_{c}}{{z}_{ip}} )}^{3} - 0.229 \cdot {( \frac{{r}_{ip}}{{z}_{ip}} )}^{3} - 0.018 \cdot {( \frac{{r}_{n}}{{z}_{ip}} )}^{3}\end{matrix} ] \cdot \frac{{F}_{z}}{π \cdot {r}_{c}^{2}} . \qquad ( 8 )$$

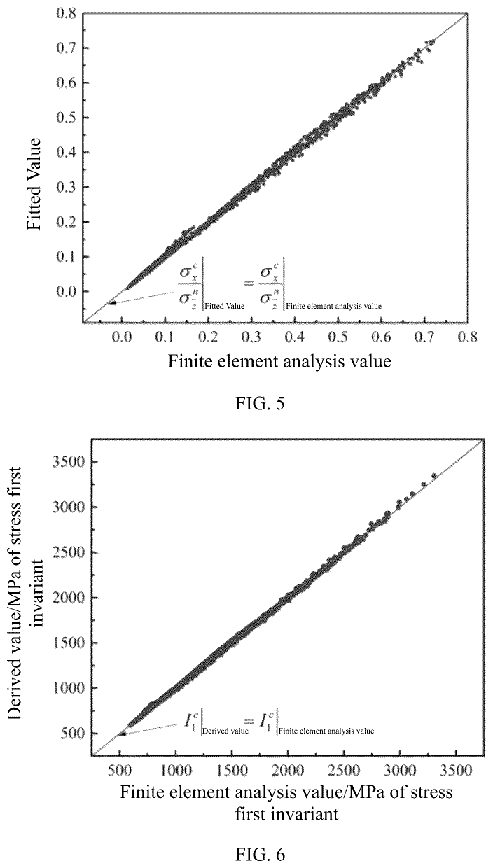

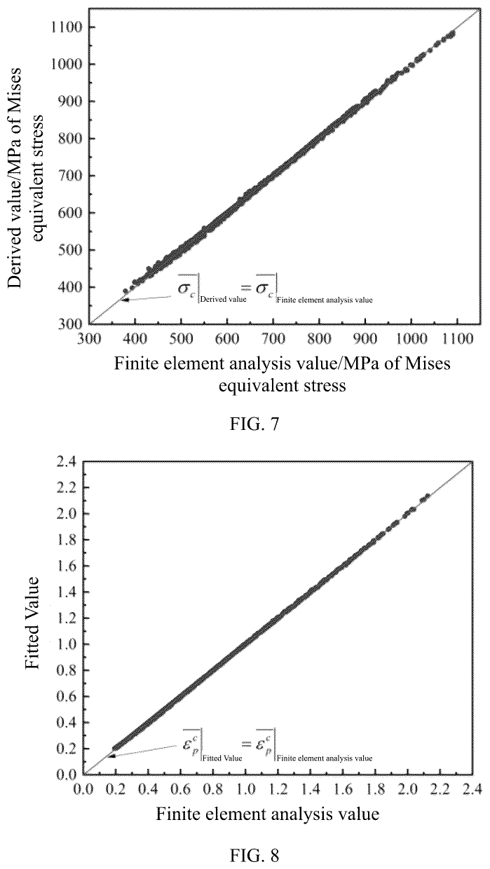

The shape characteristic parameters and acting force data of the necking specimen obtained by finite element simulation are substituted into equation (7) and equation (8) to derive the stress first invariant I₁^{c} and the Mises equivalent stress σ̅_̅{̅c̅}̅ , and the derived values of I₁^{c} and σ̅_̅{̅c̅}̅ are compared with the finite element analysis value; the results are shown in FIGS. 6 and 7, in which researchers used 1164 sets of data for corresponding comparison, and it can be seen that the derived value of I₁^{c} and the finite element analysis value are distributed centrally around the straight line I₁^{c}|_{Derived value}=I₁^{c}|_{Finite element analysis value}, and the derived value of σ̅_̅{̅c̅}̅ and the finite element analysis value are distributed centrally around the straight line σ̅_̅{̅c̅}̅ |_{Derived value}=σ̅_̅{̅c̅}̅ |_{Finite element analysis value}, showing that the necking established based on the derivation of mathematical models of stress components is minimum.

The stress first invariant I₁^{c} and the Mises equivalent stress σ̅_̅{̅c̅}̅ of the cross-section center have high accuracy with respect to mathematical models of the shape characteristic parameters r_{c}, rₙ, kₜ^{ip}, rᵢₚ, and zᵢₚ, of the necking specimen and the tensile axial acting force F_{z}.

As an example of the present invention, the following step is performed before S1:
S0: measuring an initial cross-section radius R_{c} of the specimen before the test; on the basis of S0, S1 further includes:
S11: acquiring a maximum value F_{z}^{max} of an acting force in a central axis direction and a minimum cross-section radius r_{c}⁰ of the necking bottom at the moment;
on the basis of S11, the analysis method further includes:
S4: according to F_{z}, r_{c}, rₙ, kₜ^{ip}, rᵢₚ, zᵢₚ, F_{z}^{max}, and r_{c}⁰ obtained in S1, performing a calculation on equivalent plastic strain at the center of the minimum cross-section of necking based on equation (9),

$$\overline{{ε}_{p}^{c}} = 2 ln \frac{{R}_{c}}{{r}_{c}^{0}} - \frac{{F}_{z}^{max}}{π \cdot {( {r}_{c}^{0} )}^{2} \cdot E} + {k}_{0}^{\overline{ε}} + {k}_{11}^{\overline{ε}} \cdot {k}_{t}^{ip} + {k}_{21}^{\overline{ε}} \cdot \frac{{r}_{c}}{{z}_{ip}} + {k}_{31}^{\overline{ε}} \cdot \frac{{r}_{ip}}{{z}_{ip}} + {k}_{41}^{\overline{ε}} \cdot \frac{{r}_{n}}{{z}_{ip}} + {k}_{12}^{\overline{ε}} \cdot {( {k}_{t}^{ip} )}^{2} + {k}_{22}^{\overline{ε}} \cdot {( \frac{{r}_{c}}{{z}_{ip}} )}^{2} + {k}_{32}^{\overline{ε}} \cdot {( \frac{{r}_{ip}}{{z}_{ip}} )}^{2} + {k}_{42}^{\overline{ε}} \cdot {( \frac{{r}_{n}}{{z}_{ip}} )}^{2} + {k}_{13}^{\overline{ε}} \cdot {( {k}_{t}^{ip} )}^{3} + {k}_{23}^{\overline{ε}} \cdot {( \frac{{r}_{c}}{{z}_{ip}} )}^{3} + {k}_{33}^{\overline{ε}} \cdot {( \frac{{r}_{ip}}{{z}_{ip}} )}^{3} + {k}_{43}^{\overline{ε}} \cdot {( \frac{{r}_{n}}{{z}_{ip}} )}^{3} \qquad ( 9 )$$

wherein ε̅ₚ̅^̅{̅c̅}̅ is the equivalent plastic strain at the center of the minimum cross-section of necking, E is an elastic modulus of a tensile specimen material, and k₀^{ε̅}, k₁₁^{ε̅}, k₂₁^{ε̅}, k₃₁^{ε̅}, k₄₁^{ε̅}, k₁₂^{ε̅}, k₂₂^{ε̅}, k₃₂^{ε̅}, k₄₂^{ε̅}, k₁₃^{ε̅}, k₂₃^{ε̅}, k₃₃^{ε̅}, and k₄₃^{ε̅} are equivalent strain regression coefficients.

As another example of the present invention, the following step is performed before S1:
S0′: measuring an initial cross-section radius R_{c} of the specimen before the test; on the basis of S0′, S1 further includes:
S11′: acquiring a maximum value F_{z}^{max} of an acting force in a central axis direction and a minimum cross-section radius r_{c}⁰ of the necking bottom at the moment;
on the basis of S11′, the analysis method further includes:
S4′: according to F_{z}, r_{c}, rₙ, kₜ^{ip}, rᵢₚ, zᵢₚ, and r_{c}⁰ obtained in S1, performing a calculation on equivalent plastic strain at the center of the minimum cross-section of necking based on equation (11),

$$\overline{{ε}_{p}^{c}} = 2 ln \frac{{R}_{c}}{{r}_{c}^{0}} + {k}_{0}^{\overline{ε}} + {k}_{11}^{\overline{ε}} \cdot {k}_{t}^{ip} + {k}_{21}^{\overline{ε}} \cdot \frac{{r}_{c}}{{z}_{ip}} + {k}_{31}^{\overline{ε}} \cdot \frac{{r}_{ip}}{{z}_{ip}} + {k}_{41}^{\overline{ε}} \cdot \frac{{r}_{n}}{{z}_{ip}} + {k}_{12}^{\overline{ε}} \cdot {( {k}_{t}^{ip} )}^{2} + {k}_{22}^{\overline{ε}} \cdot {( \frac{{r}_{c}}{{z}_{ip}} )}^{2} + {k}_{32}^{\overline{ε}} \cdot {( \frac{{r}_{ip}}{{z}_{ip}} )}^{2} + {k}_{42}^{\overline{ε}} \cdot {( \frac{{r}_{n}}{{z}_{ip}} )}^{2} + {k}_{13}^{\overline{ε}} \cdot {( {k}_{t}^{ip} )}^{3} + {k}_{23}^{\overline{ε}} \cdot {( \frac{{r}_{c}}{{z}_{ip}} )}^{3} + {k}_{33}^{\overline{ε}} \cdot {( \frac{{r}_{ip}}{{z}_{ip}} )}^{3} + {k}_{43}^{\overline{ε}} \cdot {( \frac{{r}_{n}}{{z}_{ip}} )}^{3} \qquad ( 11 )$$

wherein ε̅ₚ̅^̅{̅c̅}̅ is the equivalent plastic strain at the center of the minimum cross-section of necking, and k₀^{ε̅}, k₁₁^{ε̅}, k₂₁^{ε̅}, k₃₁^{ε̅}, k₄₁^{ε̅}, k₁₂^{ε̅}, k₂₂^{ε̅}, k₃₂^{ε̅}, k₄₂^{ε̅}, k₁₃^{ε̅}, k₂₃^{ε̅}, k₃₃^{ε̅}, and k₄₃^{ε̅} are equivalent strain regression coefficients.

In the prior art, the equivalent plastic strain for a uniform plastic deformation stage before uniaxial tensile necking deformation of a round bar specimen is generally approximately calculated based on equation (18),

$$2 ln \frac{{R}_{c}}{{r}_{c}^{0}} - \frac{{F}_{z}^{max}}{π \cdot {( {r}_{c}^{0} )}^{2} \cdot E} \approx 2 ln \frac{{R}_{c}}{{r}_{c}^{0}} \qquad ( 18 )$$

Therefore, equation (11) omits the elastic deformation term

$$“ \frac{{F}_{z}^{max}}{π \cdot {( {r}_{c}^{0} )}^{2} \cdot E} ”$$

with respect to equation (9), and may be regarded as an approximate calculation method of equation (9).

In this example, k₀^{ε̅}=0.108, k₁₁^{ε̅}=−0.396, k₂₁^{ε̅}=−9.768, k₃₁^{ε̅}=6.479, k₄₁^{ε̅}=2.978, k₁₂^{ε̅}=−1.193, k₂₂^{ε̅}=4.659, k₃₂^{ε̅}=−3.650, k₄₂^{ε̅}=−0.732, k₁₃^{ε̅}=1.699, k₂₃^{ε̅}=−1.076, k₃₃^{ε̅}=0.926, k₄₃^{ε̅}=0.068, and a mathematical model of the equivalent plastic strain ε̅ₚ̅^̅{̅c̅}̅ at the center of the minimum cross-section of necking obtained therefrom is shown in equation (10),

$$\overline{{ε}_{p}^{c}} = 2 ln \frac{{R}_{c}}{{r}_{c}^{0}} - \frac{{F}_{z}^{max}}{π \cdot {( {r}_{c}^{0} )}^{2} \cdot E} + 0.108 - 0.396 \cdot {k}_{t}^{ip} - 9.768 \cdot \frac{{r}_{c}}{{z}_{ip}} + 6.479 \cdot \frac{{r}_{ip}}{{z}_{ip}} + 2.978 \cdot \frac{{r}_{n}}{{z}_{ip}} - 1.193 \cdot {( {k}_{t}^{ip} )}^{2} + 4.659 \cdot {( \frac{{r}_{c}}{{z}_{ip}} )}^{2} - 3.65 \cdot {( \frac{{r}_{ip}}{{z}_{ip}} )}^{2} - 0.732 \cdot {( \frac{{r}_{n}}{{z}_{ip}} )}^{2} + 1.699 \cdot {( {k}_{t}^{ip} )}^{3} - 1.076 \cdot {( \frac{{r}_{c}}{{z}_{ip}} )}^{3} + 0.926 \cdot {( \frac{{r}_{ip}}{{z}_{ip}} )}^{3} + 0.068 \cdot {( \frac{{r}_{n}}{{z}_{ip}} )}^{3} . \qquad ( 10 )$$

Equation (9) is fitted by adopting the finite element simulation data, wherein the coefficient of determination R² after fitting adjustment is as high as 0.99993, the overall significance passes the F test (the F value is 1.325×10⁶, and the probability of being greater than F is 0), and the above 13 strain regression coefficients can all pass the t test with the significance level of 0.05, which shows that equation (10) can well describe the correlation between the equivalent plastic strain ε̅ₚ̅^̅{̅c̅}̅ at the center of the minimum cross-section of the necking and kₜ^{ip}, r_{c}/zᵢₚ, rᵢₚ/zᵢₚ, and rₙ/zᵢₚ.

The shape characteristic parameters and the acting force data of the necking specimen obtained by finite element simulation are substituted into equation (10) to carry out fitting calculation on the equivalent plastic strain ε̅ₚ̅^̅{̅c̅}̅ at the center of the minimum cross-section of the necking, and the fitted value of ε̅ₚ̅^̅{̅c̅}̅ is compared with the finite element analysis value; the results are shown in FIG. 8, and it can be seen that the data points of the fitted value of ε̅ₚ̅^̅{̅c̅}̅ and the finite element analysis value are distributed very centrally on the straight line ε̅ₚ̅^̅{̅c̅}̅|_{Fitted Value}=ε̅ₚ̅^̅{̅c̅}̅|_{Finite element analysis value}, showing that the equivalent plastic strain ε̅ₚ̅^̅{̅c̅}̅ at the center of the minimum cross-section of the necking that is calculated based on equation (10) has very high accuracy.

In another example, k₀^{ε̅}=0.108, k₁₁^{ε̅}=−0.396, k₂₁^{ε̅}=−9.768, k₃₁^{ε̅}=6.479, k₄₁^{ε̅}=2.978, k₁₂^{ε̅}=−1.193, k₂₂^{ε̅}=4.659, k₃₂^{ε̅}=−3.650, k₄₂^{ε̅}=−0.732, k₁₃^{ε̅}=1.699, k₂₃^{ε̅}=−1.076, k₃₃^{ε̅}=0.926, k₄₃^{ε̅}=0.068, and a mathematical model of the equivalent plastic strain ε̅ₚ̅^̅{̅c̅}̅ at the center of the minimum cross-section of necking obtained therefrom is shown in equation (12),

$$\overline{{ε}_{p}^{c}} = 2 ln \frac{{R}_{c}}{{r}_{c}^{0}} + 0.108 - 0.396 \cdot {k}_{t}^{ip} - 9.768 \cdot \frac{{r}_{c}}{{z}_{ip}} + 6.479 \cdot \frac{{r}_{ip}}{{z}_{ip}} + 2.978 \cdot \frac{{r}_{n}}{{z}_{ip}} - 1.193 \cdot {( {k}_{t}^{ip} )}^{2} + 4.659 \cdot {( \frac{{r}_{c}}{{z}_{ip}} )}^{2} - 3.65 \cdot {( \frac{{r}_{ip}}{{z}_{ip}} )}^{2} - 0.732 \cdot {( \frac{{r}_{n}}{{z}_{ip}} )}^{2} + 1.699 \cdot {( {k}_{t}^{ip} )}^{3} - 1.076 \cdot {( \frac{{r}_{c}}{{z}_{ip}} )}^{3} + 0.926 \cdot {( \frac{{r}_{ip}}{{z}_{ip}} )}^{3} + 0.068 \cdot {( \frac{{r}_{n}}{{z}_{ip}} )}^{3} . \qquad ( 12 )$$

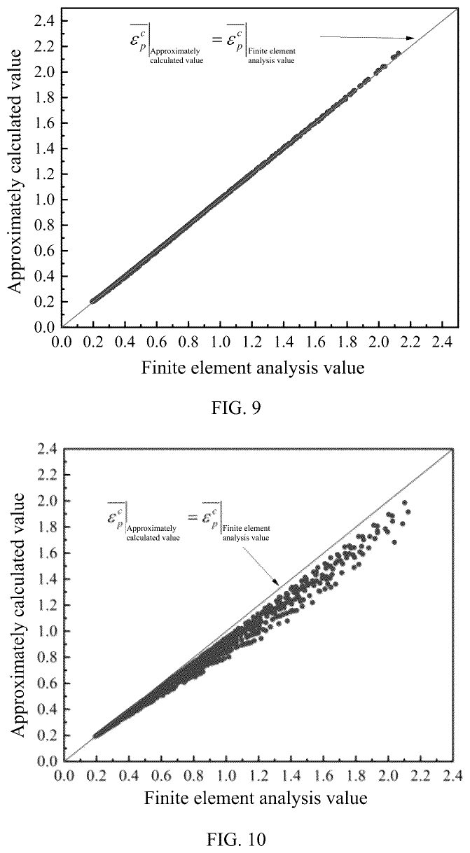

The shape characteristic parameters of the necking specimen obtained by finite element simulation are substituted into equation (12) to approximately calculate the equivalent plastic strain ε̅ₚ̅^̅{̅c̅}̅ at the center of the minimum cross-section of the necking, and the approximately calculated value of ε̅ₚ̅^̅{̅c̅}̅ obtained from equation (12) is compared with the finite element analysis value; the results are shown in FIG. 9, and it can be seen that the data points of the approximately calculated value of ε̅ₚ̅^̅{̅c̅}̅ and the finite element analysis value are distributed very centrally on the straight line ε̅ₚ̅^̅{̅c̅}̅|_{Approximately calculated value}=ε̅ₚ̅^̅{̅c̅}̅|_{Finite element analysis value}, showing that the equivalent plastic strain ε̅ₚ̅^̅{̅c̅}̅ at the center of the minimum cross-section of the necking that is approximately calculated based on equation (12) also has very high accuracy, and moreover, compared with equation (10), the detection of one parameter F_{z}^{max} can be omitted, thereby improving the detection and analysis efficiency.

In the prior art, the equivalent plastic strain ε̅ₚ̅^̅{̅c̅}̅ at the center of the minimum cross-section of the necking is generally approximately calculated based on equation (19),

$$\overline{{ε}_{p}^{c}} \approx 2 ln \frac{{R}_{c}}{{r}_{c}} \qquad ( 19 )$$

The shape characteristic parameters of the necking specimen obtained by finite element simulation are substituted into equation (19) to approximately calculate the equivalent plastic strain ε̅ₚ̅^̅{̅c̅}̅ at the center of the minimum cross-section of the necking, and the approximately calculated value of ε̅ₚ̅^̅{̅c̅}̅ obtained from equation (19) is compared with the finite element analysis value; the results are shown in FIG. 10, and it can be seen that the data points of the approximately calculated value of ε̅ₚ̅^̅{̅c̅}̅ and the finite element analysis value have a certain deviation from the straight line ε̅ₚ̅^̅{̅c̅}̅|_{Approximately calculated value}=ε̅ₚ̅^̅{̅c̅}̅|_{Finite element analysis value}. With the increase of the strain, the deviation between the data points and the straight line gradually increases, which shows that the error of the equivalent plastic strain ε̅ₚ̅^̅{̅c̅}̅ at the center of the minimum cross-section of the necking that is approximately calculated based on equation (19) in the prior art is large, and it can be seen from the comparison of FIGS. 8, 9, and 10 that the analysis and calculation precision of the equivalent plastic strain ε̅ₚ̅^̅{̅c̅}̅ at the center of the minimum cross-section provided in the present application is far higher than that in the prior art.

As an optional example, in S1, a specimen shape image is acquired during the necking deformation stage and dimension measurement and calculation are performed to obtain shape characteristic parameters, and the shape characteristic parameters obtained by the image measurement and calculation include, but are not limited to, the radius r_{c} of the minimum cross-section at the necking bottom perpendicular to the central axis on the specimen, the maximum limit value rₙ of the radius of the cross-section perpendicular to the central axis on the specimen, the tangent slope kₜ^{ip} at the inflection point position of the rotational generatrix of the necking deformation contour, the radius rᵢₚ of the cross-section perpendicular to the central axis at the inflection point position of the rotational generatrix of the necking deformation contour, and the distance zᵢₚ between the cross-section perpendicular to the central axis at the inflection point position of the rotational generatrix of the necking deformation contour and the minimum cross-section at the necking bottom. It should be noted that the means for acquiring the specimen shape image during the necking deformation stage includes, but is not limited to, photographing or recording video or other means that can be used to acquire the specimen shape image in the prior art. In addition, the above relevant parameters can be acquired by analyzing and calculating the contour during the necking deformation. As the maximum limit value rₙ of the radius of the cross-section perpendicular to the central axis on the specimen cannot be directly measured in the prior art, it can be substituted with the measured radius value at the gauge point.

In addition, a transverse extensometer can be arranged at any of the gauge end point positions on the specimen, the change situation of the diameter of the specimen is recorded by the transverse extensometer, and the maximum value F_{z}^{max} of the acting force in the central axis direction and the minimum cross-section radius r_{c}⁰ of the necking bottom at the moment are calculated and determined (the measured radius value of the cross-section at any position or gauge end point position within the gauge range at the moment on the specimen can be adopted instead).

### EXAMPLES

4 round bar specimens are adopted, wherein the specimens of Example 1, Example 3 and Example 4 are made of the same material, and the following uniaxial tensile test was performed on each round bar specimen: the initial cross-section radius R_{c} of the specimen is measured before the test; the data of loading displacement and the acting force F_{z} in the central axis is recorded in real time during the test process, and the strain perpendicular to the central axis direction is recorded by arranging a transverse extensometer at any gauge end point position on the specimen; loading is stopped at any moment during the necking deformation stage and the loading displacement is kept, the specimen is photographed, the data of the axial acting force F_{z} at the moment is recorded and then the test is completed; the maximum value F_{z}^{max} of the acting force in the central axis direction and the minimum cross-section radius r_{c}⁰ of the necking bottom at the moment are calculated and determined according to the recorded displacement, the acting force F_{z} in the central axis, the transverse strain of the specimen at the gauge end point position and the initial cross-section radius of the specimen; the necking shape characteristic parameters of the specimen contour, such as the minimum cross-section radius r_{c}, the maximum limit value rₙ of the cross-section radius, the distance zᵢₚ between the cross-section where the inflection point is located and the minimum cross-section, the radius rᵢₚ of the cross-section where the inflection point is located, and the tangent slope kₜ^{ip} at the inflection point, are measured and determined according to the specimen shape picture acquired by photographing. A first principal stress σ₁^{c}, a second principal stress σ₂^{c}, and a third principal stress σ₃^{c} at the center of the minimum cross-section of the necking bottom are calculated based on equations (3) and (4), a stress first invariant I₁^{c} at the center position of the minimum cross-section of the necking bottom is calculated based on equation (7), a Mises equivalent stress at the center position of the minimum cross-section of the necking bottom is calculated based on equation (8), and equivalent plastic strain at the center of the minimum cross-section of the necking is calculated based on equation (10) or (12).

Meanwhile, values of the first principal stress σ₁^{c}, the second principal stress σ₂^{c}, the third principal stress σ₃^{c}, the stress first invariant I₁^{c}, and the Mises equivalent stress σ̅_̅{̅c̅}̅ are acquired based on a finite element simulation mode for comparisons.

Table 1 shows the parameter information recorded when the 4 specimens are subjected to the uniaxial tensile test, the stress value and the strain value at the center of the minimum cross-section of the necking bottom calculated based on equations (3), (4), (7), (8), (10), and (12), and the stress value and the strain value at the center of the minimum cross-section of the necking bottom obtained by finite element simulation.

TABLE 1
Test detection data of examples and stress and strain values
obtained by calculation and finite element simulation values

| Examples | | | Example 1 | Example 2 | Example 3 | Example 4 |
|---|---|---|---|---|---|---|
| Elastic modulus E (MPa) of material | | | 210000 | 205000 | 210000 | 210000 |
| Initial cross-section radius R_{c} (mm) of specimen | | | 4.990 | 4.995 | 7.485 | 3.992 |
| Maximum acting force value F_{z}^{max} (N) | | | 37061 | 62869 | 83433 | 23707 |
| Minimum cross-section radius r_{c}⁰ (mm) | | | 4.631 | 4.755 | 6.947 | 3.705 |
| at the moment of maximum acting force | | | | | | |
| Acting force F_{z} (N) | | | 30971 | 51769 | 74859 | 18817 |
| Necking | Minimum cross-section | | 3.727 | 3.677 | 6.068 | 2.820 |
| shape | radius r_{c} (mm) | | | | | |
| characteristic | Maximum limit value | | 4.646 | 4.787 | 6.969 | 3.717 |
| parameter | rₙ (mm) of | | | | | |
| | cross-section radius | | | | | |
| | Distance zᵢₚ (mm) between the | | 2.831 | 2.913 | 4.973 | 2.071 |
| | cross-section where the | | | | | |
| | inflection point is located and | | | | | |
| | the minimum cross-section | | | | | |
| | Cross-section radius rᵢₚ (mm) at | | 4.033 | 4.032 | 6.397 | 3.102 |
| | the inflection point | | | | | |
| | Tangent slope kₜ^{ip} at the | | 0.162 | 0.181 | 0.098 | 0.206 |
| | inflection point | | | | | |
| Stress | First | Finite element | 832.0 | 1453.7 | 736.7 | 897.8 |
| and | principal | simulation value | | | | |
| strain | stress σ₁^{c} | (MPa) | | | | |
| at the | | Calculated value | 835.8 | 1436.9 | 738.8 | 901.2 |
| center | | of equation (1) | | | | |
| of the | | (MPa) | | | | |
| minimum | | Deviation (%) | 0.45 | −1.15 | 0.29 | 0.38 |
| cross- | Second | Finite element | 182.5 | 345.1 | 124.3 | 223.3 |
| section | principal | simulation value | | | | |
| | stress | (MPa) | | | | |
| | stress σ₂^{c} | Calculated value | 184.4 | 321.0 | 125.5 | 223.5 |
| | | of equation (2) | | | | |
| | | (MPa) | | | | |
| | | Deviation (%) | 1.00 | −6.97 | 0.95 | 0.10 |
| | Third | Finite element | 182.5 | 345.1 | 124.3 | 223.3 |
| | principal | simulation value | | | | |
| | stress | (MPa) | | | | |
| | stress σ₃^{c} | Calculated value | 184.4 | 321.0 | 125.5 | 223.5 |
| | | of equation (2) | | | | |
| | | (MPa) | | | | |
| | | Deviation (%) | 1.00 | −6.97 | 0.95 | 0.10 |
| | Mises | Finite element | 649.5 | 1108.6 | 612.4 | 674.5 |
| | equivalent | simulation value | | | | |
| | stress σ̅_̅{̅c̅}̅ | (MPa) | | | | |
| | | Calculated value | 651.4 | 1115.9 | 613.3 | 677.7 |
| | | of equation (3) | | | | |
| | | (MPa) | | | | |
| | | Deviation (%) | 0.29 | 0.66 | 0.16 | 0.47 |
| | Stress | Finite element | 1197.1 | 2143.8 | 985.3 | 1344.4 |
| | first | simulation value | | | | |
| | invariant I₁^{c} | (MPa) | | | | |
| | | Calculated value | 1204.5 | 2078.9 | 989.8 | 1348.2 |
| | | of equation (4) | | | | |
| | | (MPa) | | | | |
| | | Deviation (%) | 0.62 | −3.03 | 0.46 | 0.29 |
| | Equivalent | Finite element | 0.645 | 0.676 | 0.459 | 0.769 |
| | plastic | simulation value | | | | |
| | strain ε̅ₚ̅^̅{̅c̅}̅ | Calculated value | 0.643 | 0.680 | 0.458 | 0.766 |
| | | of equation (5) | | | | |
| | | Deviation (%) | −0.28 | 0.65 | −0.23 | −0.35 |
| | | Calculated value | 0.645 | 0.685 | 0.460 | 0.769 |
| | | of equation (6) | | | | |
| | | Deviation (%) | 0.12 | 1.29 | 0.34 | −0.01 |

Where deviation=(calculated value−finite element simulation value)/finite element simulation value×100%.

It can be seen that compared with the finite element analysis results, in the 4 examples, the maximum deviation is for the second principal stress σ₂^{c} and the third principal stress σ₃^{c} in Example 2, with the deviation of −6.97%, the rest maximum deviation is about −3%, and the deviation between most calculated values and the finite element analysis values is within 1%, so the stress and strain values at the center of the minimum cross-section in the uniaxial tensile test during the necking deformation stage of metal material round bar specimens can be accurately and effectively calculated based on the calculation method provided by the present invention.

It should be noted that before the present invention, a test method capable of measuring the stress and strain at the center position of the minimum cross-section of a specimen with necking deformation is not present in the prior art. The present invention establishes a corresponding analysis model according to the parameters, which can be measured in the test process, and achieves the measurement of the stress and strain values at the center of the minimum cross-section in the uniaxial tensile test during the necking deformation stage of a metal material round bar specimen, which is of great significance for the research of the stress-strain constitutive relation and the fracture strength of materials.

The above description is only for the purpose of illustrating the preferred embodiments of the present invention, and is not intended to limit the scope of the present invention. Any modifications, equivalents, improvements, and the like made without departing from the spirit and principle of the present invention shall fall in the protection scope of the present invention.

1. An analysis method for minimum cross-section center stress and strain of a tensile specimen with necking deformation, used for detecting and analyzing a round bar specimen, and comprising the following steps:
S1: performing a uniaxial tensile test, recording a tensile axial acting force F_{z} in a test process in real time, recording a change situation of a diameter of the specimen, and acquiring at least a radius r_{c} of a minimum cross-section at a necking bottom perpendicular to a central axis on the specimen, a maximum limit value rₙ of a radius of a cross-section perpendicular to the central axis on the specimen, a tangent slope kₜ^{ip} at an inflection point position of a rotational generatrix of a necking deformation contour, a radius rᵢₚ of a cross-section perpendicular to the central axis at the inflection point position of the rotational generatrix of the necking deformation contour, and a distance zᵢₚ between the cross-section perpendicular to the central axis at the inflection point position of the rotational generatrix of the necking deformation contour and the minimum cross-section at the necking bottom;
S2: setting a hypothetical condition according to characteristics of a contour line of necking deformation, establishing a rectangular coordinate system by taking a center position of the minimum cross-section at the necking bottom perpendicular to the central axis as an origin, taking the central axis as a coordinate z-axis, and taking any two radius lines perpendicular to each other and intersecting at a circle center within the minimum cross-section at the necking bottom as an x-axis and a y-axis of the coordinate system; and
S3: according to F_{z}, r_{c}, rₙ, kₜ^{ip}, rᵢₚ, and zᵢₚ acquired in S1, performing a calculation on a first principal stress σ₁^{c}, a second principal stress σ₂^{c} and a third principal stress σ₃^{c} at a center position of the minimum cross-section of the necking bottom based on equations (1) and (2),
$${σ}_{1}^{c} = [ \begin{matrix}{k}_{0}^{z} + {k}_{11}^{z} \cdot {k}_{t}^{ip} + {k}_{21}^{z} \cdot \frac{{r}_{c}}{{z}_{ip}} + {k}_{31}^{z} \cdot \frac{{r}_{ip}}{{z}_{ip}} + {k}_{41}^{z} \cdot \frac{{r}_{n}}{{z}_{ip}} \\ + {k}_{12}^{z} \cdot {( {k}_{t}^{ip} )}^{2} + {k}_{22}^{z} \cdot {( \frac{{r}_{c}}{{z}_{ip}} )}^{2} + {k}_{32}^{z} \cdot {( \frac{{r}_{ip}}{{z}_{ip}} )}^{2} + {k}_{42}^{z} \cdot {( \frac{{r}_{n}}{{z}_{ip}} )}^{2} \\ + {k}_{13}^{z} \cdot {( {k}_{t}^{ip} )}^{3} + {k}_{23}^{z} \cdot {( \frac{{r}_{c}}{{z}_{ip}} )}^{3} + {k}_{33}^{z} \cdot {( \frac{{r}_{ip}}{{z}_{ip}} )}^{3} + {k}_{43}^{z} \cdot {( \frac{{r}_{n}}{{z}_{ip}} )}^{3}\end{matrix} ] \cdot \frac{{F}_{z}}{π \cdot {{r}_{c}}^{2}} \qquad ( 1 )$$
$${σ}_{2}^{c} = {σ}_{3}^{c} = [ \begin{matrix}{k}_{0}^{x} + {k}_{11}^{x} \cdot {k}_{t}^{ip} + {k}_{21}^{x} \cdot \frac{{r}_{c}}{{z}_{ip}} + {k}_{31}^{x} \cdot \frac{{r}_{ip}}{{z}_{ip}} + {k}_{41}^{x} \cdot \frac{{r}_{n}}{{z}_{ip}} \\ + {k}_{12}^{x} \cdot {( {k}_{t}^{ip} )}^{2} + {k}_{22}^{x} \cdot {( \frac{{r}_{c}}{{z}_{ip}} )}^{2} + {k}_{32}^{x} \cdot {( \frac{{r}_{ip}}{{z}_{ip}} )}^{2} + {k}_{42}^{x} \cdot {( \frac{{r}_{n}}{{z}_{ip}} )}^{2} \\ + {k}_{13}^{x} \cdot {( {k}_{t}^{ip} )}^{3} + {k}_{23}^{x} \cdot {( \frac{{r}_{c}}{{z}_{ip}} )}^{3} + {k}_{33}^{x} \cdot {( \frac{{r}_{ip}}{{z}_{ip}} )}^{3} + {k}_{43}^{x} \cdot {( \frac{{r}_{n}}{{z}_{ip}} )}^{3}\end{matrix} ] \cdot \frac{{F}_{z}}{π \cdot {{r}_{c}}^{2}} \qquad ( 2 )$$
wherein σ₁^{c} is a positive stress component along the z-axis, σ₂^{c} is a positive stress component along the x-axis, σ₃^{c} is a positive stress component along the y-axis, and k₀^{z}, k₁₁^{z}, k₂₁^{z}, k₃₁^{z}, k₄₁^{z}, k₁₂^{z}, k₂₂^{z}, k₃₂^{z}, k₄₂^{z}, k₁₃^{z}, k₂₃^{z}, k₃₃^{z}, k₄₃^{z}, k₀^{x}, k₁₁^{x}, k₂₁^{x}, k₃₁^{x}, k₄₁^{x}, k₁₂^{x}, k₂₂^{x}, k₃₂^{x}, k₄₂^{x}, k₁₃^{x}, k₂₃^{x}, k₃₃^{x}, k₄₃^{x} are stress regression coefficients.

2. The analysis method for minimum cross-section center stress and strain of a tensile specimen with necking deformation according to claim 1, wherein the hypothetical condition in S2 is that: during a necking stage of the uniaxial tensile test of the round bar specimen, a shape of the specimen is a rotational body formed by rotating the rotational generatrix of the contour around the central axis; the specimen is symmetrical along a central axis direction with respect to the minimum cross-section at the necking bottom.

3. The analysis method for minimum cross-section center stress and strain of a tensile specimen with necking deformation according to claim 1, wherein k₀^{z}=1.087, k₁₁^{z}=−2.216, k₂₁^{z}=−5.935, k₃₁^{z}=5.144, k₄₁^{z}=0.432, k₁₂^{z}=0.761, k₂₂^{z}=1.828, k₃₂^{z}=−1.142, k₄₂^{z}=−0.233, k₁₃^{z}=−0.288, k₂₃^{z}=−0.483, k₃₃^{z}=0.336, k₄₃^{z}=0.026, k₀^{x}=0.059, k₁₁^{x}=−5.039, k₂₁^{x}=−11.289, k₃₁^{x}=10.229, k₄₁^{x}=0.791, k₁₂^{x}=2.439, k₂₂^{x}=2.634, k₃₂^{x}=−1.943, k₄₂^{x}=−0.397, k₁₃^{x}=−1.781, k₂₃^{x}=−0.624, k₃₃^{x}=0.565, k₄₃^{x}=0.043, and mathematical models of the first principal stress σ₁^{c}, the second principal stress σ₂^{c}, and the third principal stress σ₃^{c} obtained therefrom are shown in equations (3) and (4),
$${σ}_{1}^{c} = [ \begin{matrix}1.087 - 2.216 \cdot {k}_{t}^{ip} - 5.935 \cdot \frac{{r}_{c}}{{z}_{ip}} + 5.144 \cdot \frac{{r}_{ip}}{{z}_{ip}} + 0.432 \cdot \frac{{r}_{n}}{{z}_{ip}} \\ + 0.761 \cdot {( {k}_{t}^{ip} )}^{2} + 1.828 \cdot {( \frac{{r}_{c}}{{z}_{ip}} )}^{2} - 1.142 \cdot {( \frac{{r}_{ip}}{{z}_{ip}} )}^{2} - 0.233 \cdot {( \frac{{r}_{n}}{{z}_{ip}} )}^{2} \\ - 0.288 \cdot {( {k}_{t}^{ip} )}^{3} - 0.483 \cdot {( \frac{{r}_{c}}{{z}_{ip}} )}^{3} + 0.336 \cdot {( \frac{{r}_{ip}}{{z}_{ip}} )}^{3} + 0.026 \cdot {( \frac{{r}_{n}}{{z}_{ip}} )}^{3}\end{matrix} ] \cdot \frac{{F}_{z}}{π \cdot {{r}_{c}}^{2}} \qquad ( 3 )$$
$${σ}_{2}^{c} = {σ}_{3}^{c} = [ \begin{matrix}0.059 - 5.039 \cdot {k}_{t}^{ip} - 11.289 \cdot \frac{{r}_{c}}{{z}_{ip}} + 10.229 \cdot \frac{{r}_{ip}}{{z}_{ip}} + 0.791 \cdot \frac{{r}_{n}}{{z}_{ip}} \\ + 2.439 \cdot {( {k}_{t}^{ip} )}^{2} + 2.634 \cdot {( \frac{{r}_{c}}{{z}_{ip}} )}^{2} - 1.943 \cdot {( \frac{{r}_{ip}}{{z}_{ip}} )}^{2} - 0.397 \cdot {( \frac{{r}_{n}}{{z}_{ip}} )}^{2} \\ - 1.781 \cdot {( {k}_{t}^{ip} )}^{3} - 0.624 \cdot {( \frac{{r}_{c}}{{z}_{ip}} )}^{3} + 0.565 \cdot {( \frac{{r}_{ip}}{{z}_{ip}} )}^{3} + 0.043 \cdot {( \frac{{r}_{n}}{{z}_{ip}} )}^{3}\end{matrix} ] \cdot \frac{{F}_{z}}{π \cdot {{r}_{c}}^{2}} . \qquad ( 4 )$$

4. The analysis method for minimum cross-section center stress and strain of a tensile specimen with necking deformation according to claim 1, wherein the analysis method further comprises: calculating a stress first invariant I₁^{c} at the center position of the minimum cross-section of the necking bottom based on equations (1) and (2) in S3 as shown in equation (5)
$${I}_{1}^{c} = {σ}_{1}^{c} + {σ}_{2}^{c} + {σ}_{3}^{c} \qquad ( 5 )$$
and/or,
calculating a Mises equivalent stress σ̅_̅{̅c̅}̅ at the center position of the minimum cross-section of the necking bottom based on equations (1) and (2) in S3 as shown in equation (6)
$$\overline{{σ}_{c}} = \sqrt{\frac{{( {σ}_{1}^{c} - {σ}_{2}^{c} )}^{2} + {( {σ}_{1}^{c} - {σ}_{3}^{c} )}^{2} + {( {σ}_{2}^{c} - {σ}_{3}^{c} )}^{2}}{2}} . \qquad ( 6 )$$

5. The analysis method for minimum cross-section center stress and strain of a tensile specimen with necking deformation according to claim 4, wherein k₀^{z}=1.087, k₁₁^{z}=−2.216, k₂₁^{z}=−5.935, k₃₁^{z}=5.144, k₄₁^{z}=0.432, k₁₂^{z}=0.761, k₂₂^{z}=1.828, k₃₂^{z}=−1.142, k₄₂^{z}=−0.233, k₁₃^{z}=−0.288, k₂₃^{z}=−0.483, k₃₃^{z}=0.336, k₄₃^{z}=0.026, k₀^{x}=0.059, k₁₁^{x}=−5.039, k₂₁^{x}=−11.289, k₃₁^{x}=10.229, k₄₁^{x}=0.791, k₁₂^{x}=2.439, k₂₂^{x}=2.634, k₃₂^{x}=−1.943, k₄₂^{x}=−0.397, k₁₃^{x}=−1.781, k₂₃^{x}=−0.624, k₃₃^{x}=0.565, k₄₃^{x}=0.043, a mathematical model of the stress first invariant I₁^{c} at the center position of the minimum cross-section of the necking bottom obtained therefrom is shown in equation (7), and/or, a mathematical model of the Mises equivalent stress σ̅_̅{̅c̅}̅ at the center position of the minimum cross-section of the necking bottom obtained is shown in equation (8),
$${I}_{1}^{c} = [ \begin{matrix}1.206 - 12.294 \cdot {k}_{t}^{ip} - 28.513 \cdot \frac{{r}_{c}}{{z}_{ip}} + 25.602 \cdot \frac{{r}_{ip}}{{z}_{ip}} + 2.013 \cdot \frac{{r}_{n}}{{z}_{ip}} \\ + 5.638 \cdot {( {k}_{t}^{ip} )}^{2} + 7.095 \cdot {( \frac{{r}_{c}}{{z}_{ip}} )}^{2} - 5.027 \cdot {( \frac{{r}_{ip}}{{z}_{ip}} )}^{2} - 1.026 \cdot {( \frac{{r}_{n}}{{z}_{ip}} )}^{2} \\ - 3.85 \cdot {( {k}_{t}^{ip} )}^{3} - 1.731 \cdot {( \frac{{r}_{c}}{{z}_{ip}} )}^{3} + 1.466 \cdot {( \frac{{r}_{ip}}{{z}_{ip}} )}^{3} + 0.113 \cdot {( \frac{{r}_{n}}{{z}_{ip}} )}^{3}\end{matrix} ] \cdot \frac{{F}_{z}}{π \cdot {{r}_{c}}^{2}} \qquad ( 7 )$$
$$\overline{{σ}_{c}} = [ \begin{matrix}1.028 + 2.823 \cdot {k}_{t}^{ip} + 5.354 \cdot \frac{{r}_{c}}{{z}_{ip}} - 5.085 \cdot \frac{{r}_{ip}}{{z}_{ip}} - 0.359 \cdot \frac{{r}_{n}}{{z}_{ip}} \\ - 1.678 \cdot {( {k}_{t}^{ip} )}^{2} - 0.806 \cdot {( \frac{{r}_{c}}{{z}_{ip}} )}^{2} + 0.801 \cdot {( \frac{{r}_{ip}}{{z}_{ip}} )}^{2} + 0.164 \cdot {( \frac{{r}_{n}}{{z}_{ip}} )}^{2} \\ + 1.493 \cdot {( {k}_{t}^{ip} )}^{3} + 0.141 \cdot {( \frac{{r}_{c}}{{z}_{ip}} )}^{3} - 0.229 \cdot {( \frac{{r}_{ip}}{{z}_{ip}} )}^{3} - 0.018 \cdot {( \frac{{r}_{n}}{{z}_{ip}} )}^{3}\end{matrix} ] \cdot \frac{{F}_{z}}{π \cdot {{r}_{c}}^{2}} . \qquad ( 8 )$$

6. The analysis method for minimum cross-section center stress and strain of a tensile specimen with necking deformation according to claim 1, wherein the following step is performed before S1:
S0: measuring an initial cross-section radius R_{c} of the specimen before the test;
on the basis of S0, S1 further comprises:
S11: acquiring a maximum value F_{z}^{max} of an acting force in a central axis direction and a minimum cross-section radius r_{c}⁰ of the necking bottom at the moment;
on the basis of S11, the analysis method further comprises:
S4: according to F_{z}, r_{c}, rₙ, kₜ^{ip}, rᵢₚ, zᵢₚ, F_{z}^{max}, and r_{c}⁰ obtained in S1, performing a calculation on equivalent plastic strain at the center of the minimum cross-section of necking based on equation (9),
$$\overline{{ε}_{p}^{c}} = 2 ln \frac{{R}_{c}}{{r}_{c}^{0}} - \frac{{F}_{z}^{max}}{π \cdot {( {r}_{c}^{0} )}^{2} \cdot E} + {k}_{0}^{\overline{ε}} + {k}_{11}^{\overline{ε}} \cdot {k}_{t}^{ip} + {k}_{21}^{\overline{ε}} \cdot \frac{{r}_{c}}{{z}_{ip}} + {k}_{31}^{\overline{ε}} \cdot \frac{{r}_{ip}}{{z}_{ip}} + {k}_{41}^{\overline{ε}} \cdot \frac{{r}_{n}}{{z}_{ip}} + {k}_{12}^{\overline{ε}} \cdot {( {k}_{t}^{ip} )}^{2} + {k}_{22}^{\overline{ε}} \cdot {( \frac{{r}_{c}}{{z}_{ip}} )}^{2} + {k}_{32}^{\overline{ε}} \cdot {( \frac{{r}_{ip}}{{z}_{ip}} )}^{2} + {k}_{42}^{\overline{ε}} \cdot {( \frac{{r}_{n}}{{z}_{ip}} )}^{2} + {k}_{13}^{\overline{ε}} \cdot {( {k}_{t}^{ip} )}^{3} + {k}_{23}^{\overline{ε}} \cdot {( \frac{{r}_{c}}{{z}_{ip}} )}^{3} + {k}_{33}^{\overline{ε}} \cdot {( \frac{{r}_{ip}}{{z}_{ip}} )}^{3} + {k}_{43}^{\overline{ε}} \cdot {( \frac{{r}_{n}}{{z}_{ip}} )}^{3} \qquad ( 9 )$$
wherein ε̅ₚ̅^̅{̅c̅}̅ is the equivalent plastic strain at the center of the minimum cross-section of necking, E is an elastic modulus of a tensile specimen material, and k₀^{ε̅}, k₁₁^{ε̅}, k₂₁^{ε̅}, k₃₁^{ε̅}, k₄₁^{ε̅}, k₁₂^{ε̅}, k₂₂^{ε̅}, k₃₂^{ε̅}, k₄₂^{ε̅}, k₁₃^{ε̅}, k₂₃^{ε̅}, k₃₃^{ε̅}, and k₄₃^{ε̅} are equivalent strain regression coefficients.

7. The analysis method for minimum cross-section center stress and strain of a tensile specimen with necking deformation according to claim 6, wherein k₀^{ε̅}=0.108, k₁₁^{ε̅}=−0.396, k₂₁^{ε̅}=−9.768, k₃₁^{ε̅}=6.479, k₄₁^{ε̅}=2.978, k₁₂^{ε̅}=−1.193, k₂₂^{ε̅}=4.659, k₃₂^{ε̅}=−3.650, k₄₂^{ε̅}=−0.732, k₁₃^{ε̅}=1.699, k₂₃^{ε̅}=−1.076, k₃₃^{ε̅}=0.926, k₄₃^{ε̅}=0.068, and a mathematical model of the equivalent plastic strain ε̅ₚ̅^̅{̅c̅}̅ at the center of the minimum cross-section of necking obtained therefrom is shown in equation (10),
$$\overline{{ε}_{p}^{c}} = 2 ln \frac{{R}_{c}}{{r}_{c}^{0}} - \frac{{F}_{z}^{max}}{π \cdot {( {r}_{c}^{0} )}^{2} \cdot E} + 0.108 - 0.396 \cdot {k}_{t}^{ip} - 9.768 \cdot \frac{{r}_{c}}{{z}_{ip}} + 6.479 \cdot \frac{{r}_{ip}}{{z}_{ip}} + 2.978 \cdot \frac{{r}_{n}}{{z}_{ip}} - 1.193 \cdot {( {k}_{t}^{ip} )}^{2} + 4.659 \cdot {( \frac{{r}_{c}}{{z}_{ip}} )}^{2} - 3.65 \cdot {( \frac{{r}_{ip}}{{z}_{ip}} )}^{2} - 0.732 \cdot {( \frac{{r}_{n}}{{z}_{ip}} )}^{2} + 1.699 \cdot {( {k}_{t}^{ip} )}^{3} - 1.076 \cdot {( \frac{{r}_{c}}{{z}_{ip}} )}^{3} + 0.926 \cdot {( \frac{{r}_{ip}}{{z}_{ip}} )}^{3} + 0.068 \cdot {( \frac{{r}_{n}}{{z}_{ip}} )}^{3} . \qquad ( 10 )$$

8. The analysis method for minimum cross-section center stress and strain of a tensile specimen with necking deformation according to claim 1, wherein the following step is performed before S1:
S0′: measuring an initial cross-section radius R_{c} of the specimen before the test;
on the basis of S0′, S1 further comprises:
S11′: acquiring a maximum value F_{z}^{max} of an acting force in a central axis direction and a minimum cross-section radius r_{c}⁰ of the necking bottom at the moment;
on the basis of S11′, the analysis method further comprises:
S4′: according to F_{z}, r_{c}, rₙ, kₜ^{ip}, rᵢₚ, zᵢₚ, and r_{c}⁰ obtained in S1, performing a calculation on equivalent plastic strain at the center of the minimum cross-section of necking based on equation (11),
$$\overline{{ε}_{p}^{c}} = 2 ln \frac{{R}_{c}}{{r}_{c}^{0}} + {k}_{0}^{\overline{ε}} + {k}_{11}^{\overline{ε}} \cdot {k}_{t}^{ip} + {k}_{21}^{\overline{ε}} \cdot \frac{{r}_{c}}{{z}_{ip}} + {k}_{31}^{\overline{ε}} \cdot \frac{{r}_{ip}}{{z}_{ip}} + {k}_{41}^{\overline{ε}} \cdot \frac{{r}_{n}}{{z}_{ip}} + {k}_{12}^{\overline{ε}} \cdot {( {k}_{t}^{ip} )}^{2} + {k}_{22}^{\overline{ε}} \cdot {( \frac{{r}_{c}}{{z}_{ip}} )}^{2} + {k}_{32}^{\overline{ε}} \cdot {( \frac{{r}_{ip}}{{z}_{ip}} )}^{2} + {k}_{42}^{\overline{ε}} \cdot {( \frac{{r}_{n}}{{z}_{ip}} )}^{2} + {k}_{13}^{\overline{ε}} \cdot {( {k}_{t}^{ip} )}^{3} + {k}_{23}^{\overline{ε}} \cdot {( \frac{{r}_{c}}{{z}_{ip}} )}^{3} + {k}_{33}^{\overline{ε}} \cdot {( \frac{{r}_{ip}}{{z}_{ip}} )}^{3} + {k}_{43}^{\overline{ε}} \cdot {( \frac{{r}_{n}}{{z}_{ip}} )}^{3} \qquad ( 11 )$$
wherein ε̅ₚ̅^̅{̅c̅}̅ is the equivalent plastic strain at the center of the minimum cross-section of necking, and k₀^{ε̅}, k₁₁^{ε̅}, k₂₁^{ε̅}, k₃₁^{ε̅}, k₄₁^{ε̅}, k₁₂^{ε̅}, k₂₂^{ε̅}, k₃₂^{ε̅}, k₄₂^{ε̅}, k₁₃^{ε̅}, k₂₃^{ε̅}, k₃₃^{ε̅}, and k₄₃^{ε̅} are equivalent strain regression coefficients.

9. The analysis method for minimum cross-section center stress and strain of a tensile specimen with necking deformation according to claim 8, wherein k₀^{ε̅}=0.108, k₁₁^{ε̅}=−0.396, k₂₁^{ε̅}=−9.768, k₃₁^{ε̅}=6.479, k₄₁^{ε̅}=2.978, k₁₂^{ε̅}=−1.193, k₂₂^{ε̅}=4.659, k₃₂^{ε̅}=−3.650, k₄₂^{ε̅}=−0.732, k₁₃^{ε̅}=1.699, k₂₃^{ε̅}=−1.076, k₃₃^{ε̅}=0.926, k₄₃^{ε̅}=0.068, and a mathematical model of the equivalent plastic strain ε̅ₚ̅^̅{̅c̅}̅ at the center of the minimum cross-section of necking obtained therefrom is shown in equation (12),
$$\overline{{ε}_{p}^{c}} = 2 ln \frac{{R}_{c}}{{r}_{c}^{0}} + 0.108 - 0.396 \cdot {k}_{t}^{ip} - 9.768 \cdot \frac{{r}_{c}}{{z}_{ip}} + 6.479 \cdot \frac{{r}_{ip}}{{z}_{ip}} + 2.978 \cdot \frac{{r}_{n}}{{z}_{ip}} - 1.193 \cdot {( {k}_{t}^{ip} )}^{2} + 4.659 \cdot {( \frac{{r}_{c}}{{z}_{ip}} )}^{2} - 3.65 \cdot {( \frac{{r}_{ip}}{{z}_{ip}} )}^{2} - 0.732 \cdot {( \frac{{r}_{n}}{{z}_{ip}} )}^{2} + 1.699 \cdot {( {k}_{t}^{ip} )}^{3} - 1.076 \cdot {( \frac{{r}_{c}}{{z}_{ip}} )}^{3} + 0.926 \cdot {( \frac{{r}_{ip}}{{z}_{ip}} )}^{3} + 0.068 \cdot {( \frac{{r}_{n}}{{z}_{ip}} )}^{3} . \qquad ( 12 )$$

10. The analysis method for minimum cross-section center stress and strain of a tensile specimen with necking deformation according to claim 1, wherein in S1, a specimen shape image is acquired during a necking deformation stage and dimension measurement and calculation are performed to obtain shape characteristic parameters, and the shape characteristic parameters obtained by measurement and calculation at least comprise the radius r_{c} of the minimum cross-section at the necking bottom perpendicular to the central axis on the specimen, the maximum limit value rₙ of the radius of the cross-section perpendicular to the central axis on the specimen, the tangent slope kₜ^{ip} at the inflection point position of the rotational generatrix of the necking deformation contour, the radius rᵢₚ of the cross-section perpendicular to the central axis at the inflection point position of the rotational generatrix of the necking deformation contour, and the distance zᵢₚ between the cross-section perpendicular to the central axis at the inflection point position of the rotational generatrix of the necking deformation contour and the minimum cross-section at the necking bottom.
